(19) United States
(12) Patent Application Publication
(10) Pub. No.: US 2019/0062709 A1
(43) Pub. Date: Feb. 28, 2019

(54) METHODS AND COMPOSITIONS FOR MAINTENANCE OF HEMATOPOIETIC STEM CELLS WITH PRESERVATION OF SELF-RENEWAL CAPACITY

(71) Applicant: The Trustees of Columbia University in the City of New York, New York, NY (US)

(72) Inventors: Hans-Willem Snoeck, Brooklyn, NY (US); Larry L. Luchsinger, New York, NY (US)

(21) Appl. No.: 16/110,500

(22) Filed: Aug. 23, 2018

Related U.S. Application Data
(60) Provisional application No. 62/549,870, filed on Aug. 24, 2017.

(51) Int. Cl. C12N 5/0775

(52) U.S. Cl. CPC C12N 5/0662; C12N 2500/24; C12N 2500/16; C12N 2500/34; C12N 2500/32; C12N 2500/38; C12N 2500/12

(57) ABSTRACT

Disclosed herein are methods of culturing hematopoietic stem cells (HSCs) that enhance preservation of the HSCs self-renewal capacity and multipotency for extended periods of time. Specific embodiments comprise culturing methods that involve use of a unique low calcium media. Certain embodiments comprise methods that enable the maintenance of multipotent and self-renewing HSCs for over two weeks. Certain embodiments avoid the use of chemicals that have the risk of unexpected off-target effects or mutagenicity.

### CROSS-REFERENCE TO RELATED APPLICATIONS

The present application claims priority to U.S. Provisional Patent Application Ser. No. 62/549,870 filed Aug. 24, 2017, which is incorporated herein by reference in its entirety.

### STATEMENT OF GOVERNMENT SUPPORT

This invention was made with government support under CA167286 awarded by National Institutes of Health. The government has certain rights in the invention.

### FIELD OF THE INVENTION

The present invention relates to methods of culturing hematopoietic stem cells (HSCs) that enhance preservation of the HSCs self-renewal capacity and multipotency for extended periods of time. Specific embodiments comprise culturing methods that involve use of a unique low calcium media. Certain embodiments comprise methods that enable the maintenance of multipotent and self-renewing HSCs for over two weeks. Certain embodiments avoid the use of chemicals that have the risk of unexpected off-target effects or mutagenicity.

### BACKGROUND

Hematopoietic stem cells (HSCs) reside in the bone marrow (BM), are quiescent, can self renew, and generate all lineages of the hematopoietic system. Despite significant progress in the understanding of mechanisms involved in self-renewal, differentiation and quiescence, a coherent picture of how these mechanisms act in concert to regulate steady-state function and homeostatic responses of HSCs has not emerged yet. Furthermore, reliable renewal of HSCs in vitro has not been achieved, while there is overwhelming evidence that HSC self-renewal occurs in vivo. Two reports do suggest maintenance or expansion in vitro using small molecules. In one report, a small molecule inhibitor of the aryl hydrocarbon receptor (AHR) (stemregenin 1, SR1, a purine derivative) led to expansion of human progenitor/stem cells capable of repopulating irradiated immunodeficient mice (Boitano et al., Science 329:1345, 2011). This small molecule does not inhibit murine AHR, and does not lead to expansion of murine HSCs. Another set of molecules, pyridoindoles was also reported to expand human, but not murine HSC through a distinct mechanism ((Fares et al., Science 4345:1509, 2014).

More than 30,000 people in the US are treated with high-dose chemo and radiotherapy followed by hematopoietic rescue using cord blood, bone marrow or mobilized peripheral blood cells, which all contain HSCs. However, only a minority of eligible patients have suitable donors, either from siblings of from bone marrow or cord blood banks. In particular cord blood banking is important because of its relative technical and logistical ease. However, cord blood does not contain sufficient cells to reliably transplant adults. Procedures to expand HSCs are therefore important. Autologous HSC transplants are equally important, as they are the mainstay of salvage therapy for multiple myeloma and certain types of non-Hodgkin's lymphoma. A problem with this approach however is the contamination of the graft with patient tumor cells. Autologous transplantation used to be performed routinely in advanced stage breast cancer, but has been largely abandoned because of relapse from co-transplanted cancer cells. The ability to purify and selectively grow or even maintain HSCs while possibly depleting tumor cells would be a major advance in this setting. Finally, gene therapy targeting HSCs is an experimental treatment for immunodeficiencies and hemoglobinopathies. Transduction of the HSC with viral vectors requires in vitro culture, which invariably profoundly affects stem cell function. In this setting too, the ability to better maintain HSCs in vitro would be a major advance.

### SUMMARY OF THE INVENTION

In certain embodiments, the present invention relates to a method of maintaining hematopoietic stem cells (HSCs) in a multipotent state, the method comprising

obtaining a population of HSCs; and

culturing the population in a low calcium medium under conditions, in vitro, to maintain multipotency of the cultured population for at least 7 days, wherein the low calcium medium comprises a calcium concentration of below 1.5 mM.

In certain embodiments, the cultured population retains a capacity for self-renewal and multilineage differentiation. In additional embodiments, the calcium concentration is between about 0.01 mM and 1.5 mM. In further embodiments, the calcium concentration is between about 0.002 mM and 0.01 mM.

In additional embodiments, the cultured population exhibits Lin−Scal+Kit+CD150+ CD48−CD41−Flt3−CD34− as a surface marker profile.

In certain embodiments, culturing comprises culturing the population in a low calcium medium under conditions to maintain multipotency of the cultured population for at least 2 weeks. In certain embodiments, the low calcium medium lacks alanine, asparagine, glutamic acid or aspartic acid, or a combination thereof. In additional embodiments, the low calcium medium lacks alanine, asparagine, glutamic acid and aspartic acid.

In certain embodiments, the present invention relates to a culture medium for maintaining HSCs comprising:

one or more inorganic salts;

one or more amino acids;

one or more vitamins;

one or more saccharides;

optionally one or more trace elements, or iron selenite, insulin, transferrin, lipids and combinations thereof,

optionally one or more calpain inhibitors, and

calcium between 0.002 mM and 1.2 mM concentration.

In additional embodiments, the one or more inorganic salts comprise ferric nitrate, magnesium sulfate, potassium chloride, sodium chloride or sodium phosphate monobasic, or a combination thereof.

In further embodiments, the one or more amino acids comprise 1-arginine, 1-cystine, 1-glutamine, glycine, 1-histidine, 1-isoleucine, 1-lysine, 1-methionine, 1-phenylalanine, 1-serine, 1-threonine, 1-tryptophan, 1-tyrosine or 1-valine or a combination thereof.

In additional embodiments, the one or more vitamins comprises choline chloride, folic acid, myo-inositol, niacinamide, d-pantothenic acid, pyridoxal, riboflavin or thiamine, or a combination thereof.

In additional embodiments, the one or more saccharides is d-glucose.

In additional embodiments, the culture medium further comprises pyruvic acid.

In additional embodiments, culturing occurs for at least 14 days.

In certain embodiments, the present invention relates to a kit comprising the culture medium of described herein.

In additional embodiments, the kit further comprises one or more additional components selected from the group consisting of culture media, buffers, growth factors, and optionally cell lines.

In additional embodiments, the method further comprises culturing the cells under conditions which inhibit calpain. In additional embodiments, inhibiting calpain comprises including an inhibitor of calpain in the cell culturing conditions. In additional embodiments, the inhibitor of calpain comprises PD150606. In additional embodiments, the cells have been genetically modified to produce a reduced level of calpain.

### BRIEF DESCRIPTION OF THE DRAWINGS

The patent or application file contains at least one drawings executed in color. Copies of this patent or patent application publication with color drawing(s) will be provided by the Office upon request and payment of the necessary fee.

FIG. 1A-FIG. 1B are graphs showing Ca²⁺ signal or levels, as described. FIG. 1A; Representative live cell image traces of basal and SOCE Ca²⁺ signal in HSCs (blue), MPPs (green), CMPs (orange) stained with 1 μM FluoForte traced above perfusion scheme. Scale bars indicate relative fluorescence intensity (FΔ/Fₒ) and time (min). FIG. 1B; Quantification of basal calcium and SOCE levels within cell populations. Bars, n cells as indicated from 3 independent experiments; one-way ANOVA **P<0.05.

FIG. 2A-FIG. 2F are graphs showing various cell numbers and donor chimerism under various conditions as described. FIG. 2A; is a graph showing total cell number, and absolute number and fraction of HSCs after 2 weeks of culture in calcium-free DMEM, TPO and KL supplemented with indicated CaCl₂ concentrations. Input HSC number range (3000-4000) indicated as dotted lines. Bars: mean of cultures; n=3-7 independent experiments; one-way ANOVA *P<0.05. FIG. 2B are representative histograms (2 experiments, representative of 14 experiments) of the expression of CD41 and CD48 within the lin⁻Scal⁺ kit⁺ (LSK) gate after two weeks of culture in the presence of increasing concentration of CaCl₂. FIG. 2C is a graph showing peripheral blood donor chimerism 15 weeks after competitive transplantation of 10% of 2-week CD45.1⁺ HSC cultures together with 2×10⁵ CD45.1⁺ CD45.2⁺ competitor BM cells into CD45.2⁺ recipients. Bars, mean of recipient chimerism; n=5-15 mice from 2-3 independent experiments; one-way ANOVA ***P<0.01. FIG. 2D is a graph showing the linear correlation analysis of phenotypic HSC count at time of transplant versus corresponding 15wk donor chimerism in recipient mice. Pearson correlation; n=5-10 recipients from 2 independent experiments. FIG. 2E are graphs showing peripheral blood donor chimerism (CD45.1⁺ ) 15 weeks after secondary transplantation using 1×10⁶ BM cells from primary transplant donors into CD45.2⁺ recipients. Line graphs, ratio of donor/competitor chimerism (Δlog donor/competitor) from primary)(1°) and secondary)(2°) recipients; n=6 recipients; one-way ANOVA *P<0.05. FIG. 2F is a graph showing quantification of free [Ca²⁺ ] using the Arsenazo III method in serum and bone marrow interstitial fluid (BMIF, see methods). Bars, mean of n=6 mice; *P<0.01.

FIG. 3A-FIG. 3I. are graphs and images of differential gene expression, channels, in various cell populations and pump activity assays under various conditions. FIG. 3A is a plot showing principal component analysis of calcium gene list assembled from Gene Commons Normal Hematopoiesis module microarray data comparing HSCs (red) to 8 distinct progenitor populations (see methods). FIG. 3B is a Venn diagram of differentially expressed genes in HSCs compared to 4 broad populations; MPPb (blue), GMLPb (yellow), sCMP (green) and CLP (red) progenitor populations. Center overlap identifies a differentially expressed gene list in HSCs compared to all broad populations. Statistical analysis performed by 2-way ANOVA of population and probe expression; n=3-4 replicates per population per probe; P<0.05 considered significant. FIG. 3C is a heat map of unsupervised hierarchical clustering of differentially expressed gene list in HSCs compared to all broad populations. FIG. 3D is a graph of relative qRT-PCR expression of PMCA pumps (Atp2b1-4) and FIG. 3E is a graph of NCX exchanger channels (Slc8a1-4) in sorted cell populations. Bars; mean of individual experiments; n=6 sorted populations; one-way ANOVA *P<0.01. FIG. 3F is pan-PMCA antibody staining (glow scale) and DAPI (blue) in sorted BM populations. Scale bar is 5 μm. FIG. 3G is a graph showing quantification of relative pan-PMCA immunofluorescence intensity. Bars; mean of average of fields of 3-5 cells from 2 independent experiments; n≥30 cells; one-way ANOVA *P<0.01. FIG. 3H is a graph showing flow cytometric PMCA pump activity assay of cell populations from cKit-enriched BM cells. Non-linear regression; mean±s.e.m. fit to a one-phase decay; n=3 independent experiments. FIG. 3I is a graph showing rate constant (k) of FluoForte-bright cell decay derived from non-linear regression. Bars; mean ±s.e.m from 3 independent experiments; one-way ANOVA *P<0.01.

FIG. 4A-FIG. 4E. are graphs showing calcium concentrations, and metabolic parameters under various conditions, as well as a schematic of cellular conditions and effects of low Ca²⁺. FIG. 4A is a graph showing calcium concentration of HSCs treated for 15 min with iodoacetic acid (2 & 5 mM), oligomycin (6 & 30 μM), FCCP (1 & 5 μM) or rotenone/antimycin A (1 & 5 μM) compared to DMSO control. Bars; mean of 3 independent experiments; one-way ANOVA *P<0.05. FIG. 4B is a graph of ATP measurements from HSCs cultured for 48 h in complete media with indicated CaCl2 concentrations or freshly sorted populations. Bars; mean of 3-6 independent cultures; one-way ANOVA *P<0.05. FIG. 4C shows graphs of oxidative phosphorylation metabolic parameters derived from Seahorse Mito Stress Test using ≥5×10⁴ HSPCs (LSK+CD48−) or MPPs (LSK+CD48+ ) in DMEM containing 2.0 mM or 0.02 mM CaCl₂. Bars; mean of 4 independent experiments; two-tailed student's t-test *P<0.05, **P<0.01. FIG. 4D shows graphs of glycolytic metabolic parameters derived from Seahorse Glycolysis Stress Test using ≥5×10⁴ HSPCs (LSK+CD48−) or MPPs (LSK+CD48+ ) in DMEM containing 2.0 mM or 0.02 mM CaCl₂. Bars; mean of 4 independent experiments; two-tailed student's t-test *P<0.05, **P<0.01. FIG. 4E is a schematic representation of the mechanism of PMCA pumping activity on Ca²⁺ levels and corresponding metabolic profile.

FIG. 5A-FIG. 5D are graphs and images from various cells showing select metabolic functions and donor chimerism. FIG. 5A is a graph of Indo-1 quantification of Caᵢ²⁺ in CB and adult BM populations. Bars; n=3 independent samples; one-way ANOVA; * p<0.05. FIG. 5B are images of representative immunofluorescence of PMCA expression in CB populations (left). Quantification of PMCA immunofluorescence in sorted CB populations (right). Bars; n=2 independent experiments with 15-20 cells per experiments; one-way ANOVA; * p<0.01. FIG. 5C shows graphs of representative flow cytometric PMCA pump activity assay (top) and rate constant quantification (bottom) of cell populations from CD34-enriched CB cells. FIG. 5D shows graphs of pairwise analysis of phenotypic CB HSC counts after 7 days of culture and corresponding 8 to 9-wk donor chimerism as a function of CaCl₂ culture conditions. Line graphs; n=4-8 independent experiments; student's pairwise T-test; * p<0.05.

FIG. 6A-FIG. 6I. are graphs and a heat map reflecting various calpain or calpastatin, or MFI or Tet-1 expression on cell cultures as described. FIG. 6A is a graph showing Flow-cytometric calpain activity assay of HSCs after 24 h of culture with different concentrations of calcium in the presence of DMSO vehicle or 25 μM of the calpain-specific protease inhibitor PD150606. Bars, mean product:substrate ratio of 3 independent experiments; two-way ANOVA; *p<0.05. FIG. 6B is a graph showing long-term donor chimerism of recipient mice transplanted with 10% of 2-week HSC cultures with 2.00 mM or 0.02 mM CaCl₂ in the presence of DMSO vehicle or 25 μM of the calpain-specific protease inhibitor PD150606. Bars; mean of recipient chimerism; n=10 mice from 2 independent experiments; student's T test; *p<0.01. FIG. 6C is a plot showing flow-cytometric analysis of IRES-GFP expression in phenotypic HSCs cultured 7 days in 2.00 mM or 0.02 mM CaCl₂ DMEM media after transduction with empty vector or Cast lentiviral vectors. FIG. 6D is a graph showing donor chimerism of recipient mice 8 weeks after transplantation with 10% of empty vector or Cast-transduced cultures described in (FIG. 6C). FIG. 6E is a graph showing flow cytometric quantification of 5 hmC MFI and Tet-1 expression in BM populations after 2 weeks of culture with 2.0 mM or 0.02 mM CaCl₂ media. Bars; n=3 independent experiments, one-way ANOVA, *p<0.05. FIG. 6F is a graph showing flow cytometric quantification of TET2 expression in HSCs after 2 weeks of culture with 2.0 mM or 0.02 mM CaCl₂. Bars; n=3 independent experiments; student's t-test; * p<0.05. FIG. 6G is a graph showing quantification of percentage of phenotypic HSCs remaining in culture after 2 weeks initiated with WT or Tet2^{-/-} HSCs. Bars; n=4 independent experiments; student's t-test, *p<0.01. FIG. 6H is a graph showing donor chimerism 8 weeks post competitive transplantation using 5% of cultures from wt and Tet2^{-/-} HSC using fresh (dark grey) or 2-week cultures in 2.0 mM (light grey) or 0.02 mM (blue) CaCl₂ media. Bars; one independent transplant with 4-5 recipients per condition; two-way ANOVA; *p<0.05. Difference between wt and Tet2^{-/-} fresh HSCs: p=0.055. FIG. 6I shows graphic correlation analysis among overlapping genes between mean fold difference in calcium scRNAseq clusters and mean fold difference of Tet2 KO differential expression signature genes. Spearman r correlation test; two-tailed *p <0.05.

FIG. 7 is a table of all genes involved in Caᵢ²⁺ regulation and responses that were extracted (the ‘calciome’) from the GO database from the publicly available GEXC database (gexc.stanford.edu/models/3/genes/).

### DETAILED DESCRIPTION

Provided herein is the discovery that culturing hematopoietic stem cells (HSCs) in a low calcium media enhances preservation of the HSCs self-renewal capacity and multipotency for extended periods of time. Disclosed herein is the maintenance of multipotent and self-renewing HSCs for over two weeks. Accordingly, certain embodiments pertain to methods of culturing HSCs in vitro that provide a reliable method of maintaining HSCs for further studies. Such method embodiments avoid the use of chemicals that have the risk of unexpected off-target effects or mutagenicity.

Other embodiments pertain to novel culture medium compositions that include essential ingredients necessary for cell maintenance but which have specific, reduced levels of calcium. For example, in one embodiment, a culture medium contains the components of calcium free D9800-10 Dulbecco's MEM with calcium added at concentrations less than 1.5 mM. In a specific embodiment, the amount of calcium is between about 0.02 mM and 1.5 mM. Furthermore, in certain embodiments, it has been found that the presence of certain essential amino acids interferes with the beneficial effects of low calcium. Accordingly, in certain embodiments, low calcium medium compositions also lack of alanine, asparagine, aspartic acid and/or glutamic acid, or at least lack interfering levels of such amino acids. Interfering levels would be >0.01-0.2 mM (or >0.020 g/l) for alanine, >0.01- 0.1 mM (or >0.020 g/l) for asparagine, >0.01- 0.2 mM (or>0.060 g/l) for aspartic acid and >0.01-0.4 mM for glutamic acid. In a more specific embodiment, interfering levels (g/l) would be >0.020 g/l for alanine, >0.020 g/l for asparagine, >0.060 g/l for aspartic acid and >0.060 g/l for glutamic acid. Accordingly, culture media compositions lacking these essential amino acids would comprise less than the interfering levels.

### Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention generally are performed according to conventional methods well known in the art and as described in various general and more specific references, unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N. Y. (2000). Unless defined otherwise, all technical and scientific terms used herein have the same meaning as commonly understood by one of ordinary skill in the art.

As used herein and in the appended claims, the singular forms “a,” “an,” and “the” include plural references unless the content clearly dictates otherwise.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The “hematopoietic stem cell” refers to a cell possessing both multipotency and renewal function, which is commonly ancestor to leukocyte, erythrocyte, platelet and the like. One of the markers utilized for hematopoietic stem cells can be CD34⁺ for humans, but CD34⁻ in mice. Accordingly, in one aspect, CD34⁺ cell can be used as the hematopoietic stem cell (for human cells). In addition to CD34⁺, a plurality of other hematopoietic stem cell markers can be used in combination. Examples of human stem cell markers used in combination with CD34⁺ include CD38⁻, DR⁻, CD45⁺, CD90⁺, CD117⁺, CD123⁺, CD49f, and CD133⁺. Which stem cell marker hematopoietic cell is expressing can be determined by a method known per se such as a method using FACS, and a hematopoietic stem cell expressing a particular stem cell marker can be separated and purified.

The term “expansion” refers to increasing the number of what are called undifferentiated cells, which have not differentiated terminally, whereas the “proliferation” refers to increasing the total number of terminally differentiated cells and undifferentiated cells. The expansion of the hematopoietic cell can be evaluated by a cell marker analysis (for example, counting the cells corresponding to CD34⁺ by FACS), quantitative analysis based on the colony assay method, and the like.

The term “maintenance” refers to the culturing in vitro of hematopoietic stem cells for a period of at least 2 days wherein the cells retain their repopulation capacity, multipotency and self-renewal.

The term “low calcium media” as used herein refers to a culture media suitable for maintenance of HSCs that comprises less than 1.5 mM of calcium. Typically, the calcium level of low calcium media is less than 1 mM. In other specific embodiments, the calcium level of low calcium media is between 0.02 mM and 1 mM. In an even more specific embodiment, the calcium level is between 0.02 mM and 0.5 mM. In further embodiments, the calcium level can be any amount including and as low as 0.002 mM.

### Overview and Results

Hematopoietic stem cells (HSCs) reside in the bone marrow (BM), are quiescent, can self renew, and generate all lineages of the hematopoietic system.¹ Despite the identification of dozens of cytokines and more than 200 genes selectively or specifically regulating HSC function, a coherent picture of how these mechanisms act in concert to regulate steady-state function and homeostatic responses of HSCs has not emerged yet.- Furthermore, reliable renewal of HSCs in vitro has not been achieved while HSC self-renewal occurs in vivo.^{1, 3} From a clinical-translational point of view, it is important to bridge these gaps and devise strategies to maintain HSCs in vitro, as this would have enormous implications for the current practice of allogeneic and autologous bone marrow transplantation, as well as gene therapy targeting HSCs. A feature of HSC biology that has never been studied in detail is its calcium homeostasis, although it was suggested that a high calcium environment in the bone marrow is sensed by the Calcium sensing receptor and played a role in homing of HSCs during the perinatal transition of fetal liver to bone marrow hematopoiesis.

HSC expansion for clinical purposes, while currently experimentally attempted in dual cord blood transplantations of one fresh and one expanded unit,²⁰ has not been achieved yet. Furthermore, attempts to modify HSCs using retroviral or lentiviral transduction are hampered by loss of HSC activity during the transduction procedure, while current methods of HSC maintenance in vitro are likely insufficient to ever contemplate CRIPS/Cas9 genetic modification of HSCs.

Disclosed herein is the discovery that HSCs have strikingly low levels of cytoplasmic calcium compared to other cells, and in particular compared to progenitor cells, which are descended from stem cells but have lost their self-renewal capacity and broad differentiation potential. Bioinformatic analysis furthermore revealed that expression of dozens of genes involved in calcium entry into the cells, in shuttling of calcium between cytoplasm and organelles such as the endoplasmic reticulum and the mitochondria, and in buffering of intracellular calcium are differentially regulated in HSCs compared to progenitors and compared to mature hematopoietic of blood cells. Using currently available conditions, maintenance of HSCs in vitro has not been achieved. The cells either die, or differentiate into progenitors that cannot be transplanted to reconstitute the hematopoietic system of a lethally irradiated recipient.

Expansion of HSCs for clinical transplantation is currently not possible or clinically applied. This capability would allow much smaller donor size, for example for the use of cord blood units for transplantation into adults, and would facilitate genetic manipulation for HSC prior to transplantation, for example for the correction of immune deficiencies. Embodiments disclosed herein provide the innovative approach of utilizing a culture media with low calcium and in certain embodiments, further depleted of certain non-essential amino acids to achieve consistent expansion of HSCs and maintenance for medically significant time periods.

### Summary of Experimental Results

Based on these findings, we explored whether modifying calcium in the culture medium would affect the maintenance of HSCs in vitro. The calcium chloride concentration in classic culture media formulations range between 1.5 mM and 1.8 mM. The typical basal media for culture hematopoietic cells is DMEM or IMDM which contain 1.8 mM and 1.5 mM calcium chloride, respectively. We observed that lowering calcium in the culture media enhanced the maintenance of HSC, both as evaluated by their phenotypes and by their function in transplantation assays. Calcium concentrations were manipulated by supplementing CaCl₂ into Ca²⁺-free DMEM basal media (US Biological, cat # D9800-10), 4.5 g/L glucose, 1× StemPro34 nutrient supplement (Life Technologies), 100 ng/mL rmSCF and 30 ng/mL rmTPO (Peptrotech).

### Culture of HSCs

Hematopoietic stem cells to be used in a culture method embodiment are prepared. The animal species from which hematopoietic stem cells are derived are not particularly limited, but typically are mammals, and in particular mice and humans. The tissues from which hematopoietic stem cells are obtained are not particularly limited as long as the tissue contains hematopoietic stem cells, but typically are hematopoietic tissues. In human adults, preferred is the bone marrow, umbilical cord blood, or peripheral blood. When the tissues obtained are cell aggregates, they may be dissociated by treatment with protease, collagenase, or the like, into separated discrete cells, which may be used for culture without any further treatment. Tissues such as blood, in which cells are discrete, may be used as they are for culture without dissociation treatment of cells. Alternatively, by isolating hematopoietic stem cells from the dissociated cells, only the hematopoietic stem cells may be used.

Markers for isolating hematopoietic stem cells may be selected by a conventional way of those skilled in the art. In mice, for example, using CD34-negative, Sca-1-positive, c-Kit-positive, and lineage antigens-negative property, cell populations containing hematopoietic stem cells with long-term bone marrow-repopulating ability at a high proportion can be obtained. In the mice, hematopoietic stem cells are further defined by expression of CD150 and absence of Flt3, CD41 and CD48. Using CD34-positive, Sca-1-positive, c-Kit-positive, and lineage antigens-negative property, cell populations containing hematopoietic stem cells with short-term bone marrow-repopulating ability at a high proportion can be obtained. In humans, using CD34-positive, CD38-negative, and lineage antigen-negative property, cell populations containing hematopoietic stem cells at a high proportion can be obtained. Hematopoietic stem cells in humans furthermore express CD90 and lack CD45RA. Within the resulting cell populations, the proportion of hematopoietic stem cells may be low, but high proportion is preferred. As used herein, lineage antigens refer to marker antigens of blood cells other than hematopoietic stem cells, exemplified by a set of antigens: CD3, CD5, CD45R (B220), CD11b, Gr-1 (Ly-6 G/C), 7-4, CD41 and Ter-119. The isolation method is not particularly limited, either; cells possessing each marker property can be isolated using the antibodies, each of which has been made against each of the markers, by FACS, magnetic beads, or the like.

Exemplary of a highly enriched stem cell population is a population having the CD34⁺Thy-1⁺LIN⁻ phenotype as described in U.S. Pat. No. 5,061,620. It will be appreciated by those of skill in the art that the enrichment provided in any stem cell population will be dependent both on the selection criteria used as well as the purity achieved by the given selection techniques. Methods for isolating highly enriched populations of hematopoietic stem cells are further provided in U.S. Pat. No. 5,681,559.

In another embodiment, the cell population is initially subject to negative selection techniques to remove those cells that express lineage specific markers and retain those cells which are lineage negative (“Lin⁻”). Lin⁻ cells generally refer to cells which lack markers associated with differentiated blood cells such as T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), mast cells, eosinophils or basophils. Methods of negative selection are known in the art.

For example, the absence or low expression of markers or surface markers such as lineage specific markers can be identified by the lack of binding of antibodies specific to the marker. In one embodiment, lineage specific markers include, but are not limited to, at least one of CD2, CD14, CD15, CD16, CD19, CD20, CD38, HLA-DR and CD71; more preferably, at least one of CD14, CD15 and CD19. As used herein, “Lin⁻ refers to cells that lack expression or surface expression of at least one lineage specific marker, and typically this refers to cells that lack all lineage specific markers. Suitable lineage specific markers for human and mouse are well known in the art. Suitable Lin markers for human include CD2, CD3, CD14, CD16, CD19, CD24, CD56, CD66b, Glycophorin A. Suitable Lin markers for mouse include CD5, B220, Mac-1, Gr-1, Ter119. A cocktail of antibodies that recognize one or more the Lin markers can be used to select cells that lack expression of lineage specific markers.

In another example to separate or isolate the cells based on expression or surface expression of markers such as lineage markers, antibodies such as monoclonal antibodies can be used to identify markers associated with particular cell lineages and/or stages of differentiation. The antibodies can be attached to a solid support to such that cells that express the markers are immobilized, thereby allowing the separation of cells that express that marker from cells that do not express the marker. The separation techniques used should maximize the retention of viable cells to be collected. Such separation techniques can result in sub-populations of cells where up to 10%, usually not more than about 5%, preferably not more than about 1%, of the selected cells do not express the marker in question. The particular technique employed will depend upon efficiency of separation, associated cytotoxicity, ease and speed of performance, and necessity for sophisticated equipment and/or technical skill. An “isolated” or “purified” population of cells is substantially free of cells and materials with which it is associated in nature, in particular, free of cells that lack the desired phenotype. Substantially free or substantially purified includes at least 50% hematopoietic stem cells, preferably at least 70%, more preferably at least 80%, and even more preferably at least 90% hematopoietic stem cells.

As noted, techniques providing accurate separation of cells further include flow cytometry, which can have varying degrees of sophistication, e.g., a plurality of color channels, low angle and obtuse light scattering detecting channels, impedance channels, etc. Cells also can be selected by flow cytometry based on light scatter characteristics, where stem cells are selected based on low side scatter and low to medium forward scatter profiles. Cytospin preparations show for example, that enriched stem cells to have a size between mature lymphoid cells and mature granulocytes.

For example, in a first separation step, anti-CD34 can be labeled with a first fluorochrome, while the antibodies for the various dedicated lineages, can be conjugated to a fluorochrome with different and distinguishable spectral characteristics from the first fluorochrome. While each of the lineages can be separated (e.g., removed from the cell population) in more than one “separation” step, the lineages can be separated at the same time and/or at the same time with positive selection. The cells can be separated from dead cells, by using dyes that label dead cells (including but not limited to, propidium iodide (PI)). Separation based on negative markers, positive markers, viability and the like can be conducted separately in any order or simultaneously.

The cells described above can be used immediately or frozen at liquid nitrogen temperatures and stored for long periods of time, being thawed and capable of being reused. Once thawed, the cells can be expanded by use of the methods described herein.

In the method of expanding hematopoietic stem cells ex vivo, culturing includes any method suitable for propagating cells in vitro or ex vivo. It is understood that the descendants of the cells used to initially inoculate the culture may not be completely identical (either morphologically, genetically, or phenotypically) to the parent cell. In one embodiment, the population of cells is incubated in a suitable medium at a suitable temperature and atmosphere. The medium can be supplemented with a variety of different nutrients, heparin, antibiotics, growth factors, cytokines, and the like. In most aspects, suitable conditions comprise culturing at about 33° C. to about 39° C., and preferably at about 37° C. In one embodiment the oxygen concentration is about 4 to about 20%. The medium can be replaced throughout the culture period. In one preferred embodiment, half of the medium is replaced twice per week with fresh media.

The population of cells is placed in a suitable container for expanding the HSCs. For example, suitable containers for culturing the population of cells include flasks, tubes, or plates. In one embodiment, the flask can be T-flask such as a 12.5 cm², or a 75 cm² T-flask. The plate can be a 10 cm plate, a 3.5 cm plate or a multi-welled plate such as a 12, 24, or 96 well plate. The wells can be flat, v-bottom or u-bottom wells. The containers can be treated with any suitable treatment for tissue culture to promote cell adhesion or to inhibit cell adhesion to the surface of the container. Such containers are commercially available from Falcon, Corning or Costar. As used herein, “expansion container” also is intended to include any chamber or container for expanding cells whether or not free standing or incorporated into an expansion apparatus.

Preferably, the cell density of the cultured population of cells such as total bone marrow is from at least about 1×10² cells to about 1×10⁷cells/mL, and even more preferably from about 1×10⁵ to about 1×10⁶ cells/mL, and cells are cultured at an oxygen concentration of from about 2 to 20%. In one embodiment, SP bone marrow cells are cultured a lower density, for example from about 1×10² to 5×10³ cells/ml. In a separate aspect, the inoculation population of cells is derived from mobilized peripheral blood and is from about 20,000 cells/mL to about 50,000 cells/mL, preferably 50,000 cells/mL.

Any of the above-mentioned culture methods may include the step of isolating CD-34-negative, Sca-1-positive, c-Kit-positive, and lineage-antigen-negative hematopoietic stem cells from bone marrow cells or blood cells from mice. The aforementioned lineage antigen may be a set of antigens consisting of CDS, CD45R (B220), CD11b, Gr-1 (Ly-6G/C), 7-4, and Ter-119.7.

A conventional medium for culturing hematopoietic cells is a basal medium further including specific ingredients required for culturing hematopoietic cells. However, the calcium content of the basal medium is controlled below certain thresholds, typically below 1.2 mM, below 1.0 mM or below 0.5 mM calcium. The basal medium may be a serum-free medium or a serum-containing medium. The serum-free medium may be one that includes the ingredients of Iscove's MDM (IMDM), but where calcium levels are below 1.2 mM and optionally, lack certain essential amino acids. One option that works well is DMEM (Dulbecco's Modified Eagle Medium). The basal medium may be commercially available, and the composition thereof may be obvious to those of skilled in the art. For example, the composition of IMDM is disclosed in U.S. Pat. No. 5,945,337. The basal medium is prepared to provide i) inorganic salts (e.g., potassium, calcium (at controlled levels), phosphate, etc.) to maintain cell osmolality and mineral requirements, ii) certain essential amino acids, iii) carbon sources such as glucose which is used for cell energy metabolism, and iv) various vitamins and cofactors which can be required in cell growth such as riboflavin, nicotinamide, folic acid, choline, and biotin. Glutamine is one of essential amino acids that can be added to the medium of an embodiment of the present invention in an effective amount. The concentration of glutamine may be from 100 to 500 μg/ml, preferably from 125 to 375 μg/ml, and more preferably from 150 to 300 μg/ml. Glutamine is sometimes added to the medium immediately before the use of the medium due to its instability. A buffer which maintains the pH of the medium during the cell metabolism may be added to the basal medium. In general, the buffer may be a bicarbonate or HEPES. The pH of the basal medium is generally in the range of 6.8 to 7.2.

According to an embodiment of the present invention, there is provided a method of culturing hematopoietic cells including: introducing the hematopoietic cells into a culture container including the medium for culturing hematopoietic cells according to an embodiment of the present invention; and culturing the hematopoietic cells.

### EXAMPLES

### Example 1

### Low Calcium Media Formulation

Media Formulation
Components shown as gaiter. The values below may be adjusted 0-50% +/− the stated amount.

### Inorganic Salts

Ferric Nitrate.9H₂O 0.0001
Magnesium Sulfate 0.09767
Potassium Chloride 0.40
Sodium Chloride 6.40
Sodium Phosphate Monobasic 0.109

### Amino Acids

L-Arginine.HCl 0.084
L-Cystine.2HCl 0.0626
L-Glutamine 0.584
Glycine 0.030
L-Histidine.HCl.H₂O 20 0.042
L-Isoleucine 0.105
L-Leucine 0.105
L-Lysine.HCl 0.146
L-Methionine 0.03
L-Phenylalanine 0.066
L-Serine 0.042
L-Threonine 0.095
L-Tryptophan 0.016
L-Tyrosine.2Na.2H₂O 0.10379
L-Valine 0.094

### Vitamins

Choline Chloride 0.004
Folic Acid 0.004
myo-Inositol 0.0072
Niacinamide 0.004
D-Pantothenic Acid, Na 0.004
Pyridoxal.HCl 0.004
Riboflavin 0.0004
Thiamine.HCl 0.004

### Other

D-Glucose 1.00
Phenol Red, Sodium 0.0159
Pyruvic Acid, Sodium 0.11

### Example 2

### Low Calcium Media Formulation

The values provided below for concentration or moles may be +/−10-50% for each of the stated values, with the exception of calcium chloride, which may be the stated value or less. One or more of L-Alanine, L-Asparagine, L-Aspartic acid and L-Glutamic acid are lacking from the formulation.

| | Molecular | Concentration | |
|---|---|---|---|
| Components | Weight | (mg/L) | mM |
| Amino Acids | | | |
| Glycine | 75.0 | 30.0 | 0.4 |
| L-Alanine (if present) | 89.0 | 25.0 | 0.28089887 |
| L-Arginine hydrochloride | 211.0 | 84.0 | 0.39810428 |
| L-Asparagine (freebase) (if present) | 132.0 | 25.0 | 0.18939394 |
| L-Aspartic acid (if present) | 133.0 | 30.0 | 0.22556391 |
| L-Cystine 2HCl | 313.0 | 91.4 | 0.29201278 |
| L-Glutamic Acid (if present) | 147.0 | 75.0 | 0.5102041 |
| L-Glutamine | 146.0 | 584.0 | 4.0 |
| L-Histidine hydrochloride-H₂O | 210.0 | 42.0 | 0.2 |
| L-Isoleucine | 131.0 | 105.0 | 0.8015267 |
| L-Leucine | 131.0 | 105.0 | 0.8015267 |
| L-Lysine hydrochloride | 183.0 | 146.0 | 0.7978142 |
| L-Methionine | 149.0 | 30.0 | 0.20134228 |
| L-Phenylalanine | 165.0 | 66.0 | 0.4 |
| L-Proline | 115.0 | 40.0 | 0.3478261 |
| L-Serine | 105.0 | 42.0 | 0.4 |
| L-Threonine | 119.0 | 95.0 | 0.79831934 |
| L-Tryptophan | 204.0 | 16.0 | 0.078431375 |
| L-Tyrosine disodium salt | 225.0 | 104.0 | 0.46222222 |
| L-Valine | 117.0 | 94.0 | 0.8034188 |
| Vitamins | | | |
| Biotin | 244.0 | 0.013 | 5.327869E−5 |
| Choline chloride | 140.0 | 4.0 | 0.028571429 |
| D-Calcium pantothenate | 477.0 | 4.0 | 0.008385744 |
| Folic Acid | 441.0 | 4.0 | 0.009070295 |
| Niacinamide | 122.0 | 4.0 | 0.032786883 |
| Pyridoxal hydrochloride | 204.0 | 4.0 | 0.019607844 |
| Riboflavin | 376.0 | 0.4 | 0.0010638298 |
| Thiamine hydrochloride | 337.0 | 4.0 | 0.011869436 |
| Vitamin B12 | 1355.0 | 0.013 | 9.594096E−6 |
| i-Inositol | 180.0 | 7.2 | 0.04 |
| Inorganic Salts | | | |
| Calcium Chloride (anhyd.) | 111.0 | 165.0 | 1.4864864 |
| Magnesium Sulfate (anhyd.) | 120.0 | 97.67 | 0.8139166 |
| Potassium Chloride (KCl) | 75.0 | 330.0 | 4.4 |
| Potassium Nitrate (KNO₃) | 101.0 | 0.076 | 7.524752E−4 |
| Sodium Bicarbonate (NaHCO₃) | 84.0 | 3024.0 | 36.0 |
| Sodium Chloride (NaCl) | 58.0 | 4505.0 | 77.67242 |
| Sodium Phosphate monobasic | 138.0 | 125.0 | 0.9057971 |
| (NaH₂PO₄—H₂O) | | | |
| Sodium Selenite | 173.0 | 0.017 | 9.8265904E−5 |
| (Na₂SeO₃—5H₂0) | | | |
| Other Components | | | |
| D-Glucose (Dextrose) | 180.0 | 4500.0 | 25.0 |
| HEPES | 238.0 | 5958.0 | 25.033613 |
| Phenol Red | 376.4 | 15.0 | 0.039851222 |
| Sodium Pyruvate | 110.0 | 110.0 | 1.0 |

### Example 3

### Expansion and Maintenance of HSCs with low Ca²⁺ media

### Animals

C57BL/6J mice (CD45.2) and B6.SJL-Ptprca^{PeP3b/BoyJ}(CD45.1) were purchased from The Jackson Laboratory (Bar Harbor, Me.). CD45.12 competitor mice were produced in house by pairing CD45.2 and CD45.1 breeders. Conditional Mito-Dendra2 transgenic (Pham) mice(Pham et al., 2012) (B6;129S-Gt(ROSA)26Sor^{tml(CAG-COX8A/Dendra2)Dcc/J}) and E2A-Cre mice³³ (B6.FVB-Tg(EIIa-cre)C5379Lmgd/J)(Lakso et al., 1996) were purchased from Jackson Laboratory. Briefly, Pham mice contain a mitochondrial signal peptide (CoxVIII) fused to the Dendra photoactivatable fluorescent reporter cDNA preceded by a stop-lox sequence in the Rosa locus. These mice were crossed with E2A-Cre mice to effect ubiquitous induction of the Mito-Dendra2 reporter. Animals were housed in a specific pathogen-free facility. Experiments and animal care were performed in accordance with the Columbia University Institutional Animal Care and Use Committee. All mice used were between 8-12 weeks of age. Both sexes were used for experiments. Results were analyzed in non-blinded fashion. In all experiments, randomly chosen littermates were used.

### Human Cord Blood and BM Samples

De-identified cord blood was obtained from the New York Blood Center, de-identified BM samples from the leftover collections bags from the Columbia Bone Marrow Transplantation program. The experiments were performed in accordance with protocol approved by Columbia University Institutional Review Board protocol number AAAR0324.

### Cell Lines

NIH-3T3 cells were purchased from ATCC (Manassas, Va.) and sub-cultured in 10% 10% calf serum/DMEM with penicillin/streptomycin and split 1:3 every 3 days. All lines are tested yearly for Mycoplasma contamination and found negative.

### FACS Sorting and Analysis

For peripheral blood analyses, erythrocytes were lysed twice with ACK lysis buffer and nucleated cells were stained with antibody cocktail (Supplementary Table 2) in FACS buffer for 15 min on ice, washed and analyzed on a BD Fortessa flow cytometer (Becton Dickinson, Mountain View, Calif.). For bone marrow analyses, cells were isolated using the crushing method and erythrocytes were lysed with ACK lysis buffer followed by 40 μm filtration. BM cells were stained with antibody cocktail in FACS buffer for 30 min on ice, washed and analyzed on a BD LSRII flow cytometer (Becton Dickinson, Mountain View, Calif.). Dead cells were excluded from analyses by gating out 7AAD-positive cells. To isolate purified hematopoietic populations, BM cells were isolated, stained and sorted using a BD Influx cell sorter (Becton Dickinson, Mountain View, Calif.) into complete media. Data were analyzed using FlowJo9.6 (TreeStar Inc., Ashland, OR) and FCS Express 6 (DeNovo Software, Los Angeles, Calif.).

### Hematopoietic Stem Cell Culture

Long term LT-HSCs cultures were carried out using Ca²⁺-free DMEM (U.S. Biologicals) reconstituted with 4.5 g/L glucose and 1.1 g/L sodium bicarbonate at pH 7.4. Complete media was prepared with Ca²⁺-free DMEM supplemented with 1× StemPro34 SFM (Invitrogen), 10 mM HEPES, 200 ng/mL of recombinant m-SCF and 30 ng/mL m-TPO (Peptrotech, Rocky Hill, N.J.). Media of various calcium concentrations was prepared as serial dilutions from stock complete media at 2 mM. Cultures were initiated with 3000-4000 purified HSCs (LSK+CD48−F1t3−CD150+) and cultured in round bottom 96 well plates with 150 uL of media in 5% O₂ at 37° C. Half of the media volume was changed every 3 days and split 1:2 every 7 days. Cultures were counted at the time of phenotypic flow cytometric percentage analysis to determine phenotypic HSC counts at the end of culture. Based on calculations, HSC yields were expressed as phenotypic HSC at the end of culture divided by phenotypic HSC count at the start of culture.

### Hematopoietic Stem Cell Transplantation

All transplant experiments were initiated with LT-HSC cultures consisting of CD45.1 donor HSCs. At completion of culture, 10% percent of cultures at various Ca²⁺ concentrations were mixed with 200K total BM cells from CD45.12 competitors for and transplanted into CD45.2 mice lethally irradiated with a split dose totaling 9.75 Gy of X-ray irradiation. For competitive transplantation experiments, at least two independent transplants, each with at least 4 recipients per condition of Ca²⁺ culture were performed, and result of all recipients pooled for statistical analysis. Sample sizes were estimated from power calculations based on results of the preliminary experiment.

### Seahorse Metabolic Flux Experiments

For all Seahorse metabolic assays, Ca2+-free DMEM media was prepared without sodium bicarbonate or HEPES buffer and neutralized to pH 7.4 at 37° C. (Buffer-free DMEM) immediately prior to the experiments. XFp flux cartridges were hydrated in XF Calibrant overnight. For all assays, a pool of (LSK+CD48−) cells or MPPs (LSK+CD48+) cells were isolated from 6 mice and ≥50,000 cells plated onto one well of a Seahorse XFp culture plate coated overnight with Cell-Tak reagent. Cells were immobilized by centrifugation at 200×g for 5 min at RT and washed twice with either 2.0 mM or 0.02 mM CaCl₂ Buffer-free DMEM. Cells were equilibrated in fresh 2.0 mM or 0.02 mM CaCl₂ Buffer-free DMEM in a humidified non-CO₂ incubator until the start of the assay. Flux cartridges were loaded with drugs prepared with Buffer-free DMEM according to manufacturer's instructions. Oxidative phosphorylation and glycolysis values were obtained using the XFp Mito Stress Kit and Glycolysis Stress Kit, respectively. Metabolic parameters were derived from calculations based on manufacturer's instructions. Due to feasibility of cell number isolation, experiments are represented as one technical replicate per cell type, per condition over 4 independent experiments.

### Quantitative RT-PCR

Sorted or cultured cell populations (2-5×10³ cells) were lysed in Trizol LS reagent (Invitrogen, Carlsbad, Calif.) and RNA was isolated according to manufacturer's instructions. cDNA was synthesized using Superscript III Reverse Transcriptase (Invitrogen) and target CT values were determined using inventoried Taqman probes (Applied Biosystems, Carlsbad, Calif., see Supplementary Table 3) spanning exon/exon boundaries and detected using a Viia7 Real Time PCR System (Applied Biosystems). Relative quantification was calculated using the AACT method. All values were normalized to relative multiplexed GAPDH-VIC values.

### Indo-I-AM Calcium Flux

BM was freshly isolated and lineage depleted with the MACS Lineage Depletion Kit or cKit enriched using anti mouse-cKit-biotin with Streptavidin-microbeads (Miltenyi Biotech, San Diego, Calif.) according to manufacturer's instructions. Cells were incubated at 1×10⁶ cells/mL in complete medium supplemented with 2 μM Indo-1 prepared as stock supplemented with Pluronic-F127 and incubated at 37° C. for 15 min. Cells were washed and stained for surface markers for 10 min at 25° C., washed and allowed to rest for 15 min in PBS with Ca²⁺. FACS tubes were run at 37° C. in the sample port of the LSRII flow cytometer equipped with a 355nM excitation laser. Filter sets corresponding to violet emission (379±15 nm) and blue emission (515±20 nm) were used to report Ca²⁺ bound and free dye ratio, respectively. Events were collected for 40 seconds prior to incubation with 1 uM SDF1 (Peprotech) to induce calcium transients. The average ratio, R, of bound/free Indo-1 (405 nm/485 nm emission) before simulation was used to determine baseline values. Identical samples were equilibrated in 10 mM EDTA PBS w/o Ca2+ to determine Rₘᵢₙ or stimulated with 1 uM ionomycin to determine Rₘₐₓ. The Indo-1 dissociation constant (K_{d}) was assumed to be 237 nM at 37° C. based on previous studies.⁴ The following equation was then used to relate Indo-1 intensity ratios to [Caᵢ²⁺] levels;

$$[ {Ca}^{2 +} ] = {K}_{d} \cdot \frac{( R - {R}_{min} )}{( {R}_{max} - R )}$$

### Calcium Imaging

Fresh sorted HSCs, MPPs and MPs were plated on 35mm glass bottom dishes (MatTek Corporation, P35G-1.5-7-C) coated with Cell-Tak according to manufacturer's instructions. Cells were washed with media containing classical CaCl₂ formulation (1.8 mM) in the presence of 1:1,000 FluoForte and stained for 15 min at RT. Baseline fluorescence intensity was collected for 5 min and then cells were perfused with 5 mM EGTA for 5 min to chelate extracellular calcium. The cells were then perfused with media 2 mM thapsigargin/5 mM EGTA for 5 min to induce ER store depletion after which cells were perfused with fresh media to induce store operated calcium entry (SOCE) and restoration of calcium fluorescence to basal levels of cytoplasmic calcium ions. Immunofluorescence images were collected at 1 frame per 5 sec and imaged using a custom Nikon microscope Ti-E (Andor Zyla sCMOS camera; Nikon CFI Plan Apo Lambda, 20×/0.75/1.0mm) with automated motorized stage and environmental controls set at 5% CO₂, 20% O₂ and 37° C. (TOKAI HIT) and a standard GFP/FITC filter (Nikon, # 96362, ET Sputter Coat, Ex470/40 Dm495 Bar525/50). Basal level of cytoplasmic calcium ions and SOCE were analyzed using NIS Element (Nikon) and Prism (Graphpad). The average of 10-20 cells was collected in each of three independent experiments.

### Immunofluorescence

Freshly isolated or cultured hematopoietic populations (2-5×10³ cells) were collected in complete media and plated on onto MicroWell 96-well glass-bottom plates (Thermo, Waltham, Mass.) coated with 1μg/mL poly-D-lysine or 30 μg/cm2 Cell-Tak (Corning, Bedford, Mass.). Cells were allowed to adhere for 10 min and fixed with 4% PFA/PHEM buffer for 15 min. Cells were then permeabilized with 0.1% TritonX-100/PBS for 5 min and blocked with 2% BSA/PBS for lh at 4° C. Cells were incubated with anti-pan-PMCA (1:250) overnight, washed and incubated with AlexaFluor secondary antibodies (Invitrogen) for lh. Cell nuclei were counterstained with DAPI or DRAQ5 and mounted with fluorescent mounting media (Vector Labs, Burlingame, Calif.). Images were acquired with a Leica TCS SP8 confocal microscope or a Leica DMI 6000B and deconvoluted and processed with Leica AF6000 software package.

### Live Cell Mitochondria Imaging

For mitochondrial live cell imaging, confocal XY-time series stacks (1.5 0 m focal plane, 40× 1.1 NA objective) were collected and processed using Imaris surface module (Bitplane, Belfast, UK). Individual mitochondria surfaces were automatically traced and corresponding tracks were thresholded for a quality score of >15 and a duration >20 seconds. Automated mitochondrial surface track calculations were manually inspected and edited as appropriate to reflect mitochondrial movement. Ambiguous tracks or aberrant movements from automatic track calculations were edited as appropriate. Measurements of thresholded mitochondrial track velocity, duration, track length and displacement were collected. The mean±SEM number of mitochondria falling into each velocity and length category collected from 10 cells each from 3 experiments were expressed.

For photoconversion of mito-Dendra2, still frames where acquired of sorted populations of cells as described above and imaged on a Leica SP8 confocal microscope set to bleachpoint mode. ROIs were selected corresponding to individual mitochondria and photoconversion was obtained by applying 30% 488 nm laser light for 30-50 ms dwell time. Live cell imaging was immediately resumed collecting at 515±25 nm for Dendra2 and 560±30 nm for photoconverted Dendra2.

### FluoForte Stainin, PMCA Activity Assa, and Calpain Activity Assay

PMCA assay were performed as previously described with slight modification.(de Jong and Kuypers, 2007) Briefly, cKit-eriched cells were stained with FluoForte at 1×10⁶/mL in Buffer B (10 mM HEPES pH 7.4, 145 mM NaCl, 7 mM KCl, 0.1 mM EGTA, 0.15 mM MgCl₂, 5 mM glucose, 5 mM inosine, 5 mM pyruvate) at 1:1000 dilution for 15 min at 37° C. Cells were washed in Buffer B and stained with flow cytometric antibodies for 15 min at RT. Cells were loaded with Ca²⁺ in incubated in HBSF Buffer (10 mM HEPES pH 7.4, 145 mM NaCl, 0.15 mM MgCl₂, 5 mM glucose, 5 mM inosine) containing 25 μM CaCl₂ for 5 min at 37° C., followed by addition of 800 nM A23187 calcium ionophore to equalize intracellular and extracellular calcium for 1 min. A23187 was quenched by addition ice cold 2% BSA in HBSF Buffer and immediately placed on ice until start of assay. To induce PMCA activity, cells were added to 37° C. water bath and acquired in a heated sample port of a BD LSRII cytometer at various time points up to 20 min. The percentage of FluoForte bright cells was analyzed in HSC, MPP and CMP compartments. The decrease in fraction of FluoForte bright cells was plotted versus time and fitted to a one-phase decay regression using Prism 7. FluoForte decay rate constant values were measured and the mean of 3 independent experiments were calculated.

For Calpain activity assay, sorted cells were incubated with tBOC-Leu-Met-CMAC (Thermo Fisher) at 1 μM for 15 min at 37° C. in the presence of DMSO or 10 μM PD150606 calpain inhibitor (PD10606 is: (Z)-3-(4-Iodophenyl)-2-mercapto-2-propenoic acid, available from Tocris Bioscience (Bristol, United Kingdom), or as CAS 426821-41-2 from EMD Millipore). Flow cytometric analysis with excitation at 355 nm was used to collect substrate (˜405 nm) and product (˜450 nm) emission. MFI was then calculated and product/substrate ratio was calculated. All ratios were normalized to PD1506060 control.

### BM Interstitial Fluid Calcium Quantification

Mice 8-10 week old were euthanized with CO₂ followed by cervical dislocation. A cardiac puncture was performed to collect blood for serum as a Ca²⁺ measurement control. To collect serum, peripheral blood was isolated without chelation and allowed to coagulate for 20 min at RT followed by centrifugation at 400×g for 10 min. Serum was isolated from the top phase and stored at 4° C. Tibias were removed of all muscle and connective tissue and the bones were cut near the epiphysis on both sides at an angle to expose the BM cavity. Bones were placed in a 1.5 mL eppendorf tube and centrifuge at 400×g for one min. Bones were removed and an estimate of the BM volume was measured, which was consistently 20-250 μL between mice. Bone marrow was isolated without chelation and allowed to coagulate for 20 min at RT followed by centrifugation at 400×g for 10 min. A thin film of fluid was visible on top of the pellet and it was estimated there was ˜1 uL of interstitial fluid which was extracted by adding water at the estimated volume of BM at isolation. The tube was tapped to gently mix and allowed to equilibrate for 5 min. Samples were centrifuged at 400×g for 5 min and supernatant was removed. The entire volume was place in a 96 well along with corresponding dilutions of serum and standards. To measure Ca²⁺ concentration, 100 μL of Arsenazo reagent was added to each well and absorbance was measured at 660 nm. Ca²⁺ concentrations were calculated based on absorbance and dilution factor by using the linear regression of a standard curve generated using 1:2 dilutions ranging from 5 mM to 5□M CaCl₂ prepared in diH₂O.

### Gene Expression Commons Bioinformatics Analysis

Microarray data was downloaded for the Gene Expression Commons website https.//gexc.riken.jp) using the Mouse Hematopoiesis module.(Seita et al., 2012) The complete gene expression activity list for each progenitor population dataset of interest were collated into a single file. A list of genes annotated to be involved in calcium signaling (i.e. Calciome) was assembled from the KEGG Pathway Database (www.genome.jp/kegg/patinway.html) and probes corresponding to genes in the Calciome gene list.(Arakawa et al., 2005; Ogata et al., 1998) Probes with high dynamic range were selected to represent genes and unsupervised hierarchical clustering was performed using the FluidigmSC script using R program. Two-way ANOVA analysis of the selected gene probes were used to identify significant differentially expressed genes between HSCs and other populations using Prism 7. For Venn diagram analysis, gene lists identified as statistically different in HSCs compared to MPPb, GMLPb, sCMP and CLP populations (i.e. progenitor populations) were assembled and visualized using Venny 2.0 (littp://bioinfogp.cnb.csic.ec/tools/vennyl). To assess the accuracy of selected probes, all probe sets corresponding to genes listed as statistically different between HSCs and progenitor populations from the selected probe list were analyzed for statistical significance between populations and the percentage of probes reaching statistical significance were reported.

A relative expression analysis of the calciome gene list compared to the entire transcriptome and the genes identified as contributing a severe defect to HSC function (i.e. severe defect genes) in knockout models was also performed. For box, scatter and whisker plotting, R version 3.2.5 was used on the three gene sets (R Core Team (2016). R: A language and environment for statistical computing. R Foundation for Statistical Computing, Vienna, Austria) (https://www.R- project.org), along with the package ggplot2 and ggrepel(Wickham, 2009) (https://CRAN.Rproject.org/package=ggrepel).

### Single Cell RNA Sequencing

Briefly, 96 phenotypic single cell HSCs were index sorted into lysis buffer from 2.0 mM and 0.02 mM CaCl₂ cultures 14 days after initiation of culture. After sorting, plates were spun down, frozen, and delivered to the Columbia University Genomic Center Single Cell Analysis Core and processed with automated liquid handling. Briefly, template-switching reverse transcription was performed with adapter-linked oligo(dT) primers containing both cell- and molecule-specific barcodes, and cDNAs were pooled for PCR and library construction. Pooled, 3′-end sequencing libraries were then sequenced on an Illumina NextSeq 500. Differential gene expression was carried out in R project software using Seurat package. Cells were assigned to clusters using multidimensional analysis of gene expression. Significantly differential genes within clusters were assigned with a FDR <0.1 and p <0.05.

### Image Quantification

For PMCA immunofluorescence intensity measurements, confocal or deconvoluted z-stacks were collected and projected as a z-project in ImageJ. Thresholds were set based on IgG-stained negative control cells and the integrated density value of each signal per cell was recorded. The mean±SEM for 15 fields (30-50 cells) are expressed.

### Calpain Activity Assay

The live cell calpain activity assay were carried out as previously described with modifications(Niapour and Berger, 2007). Purified HSCs were cultured at various CaCl₂ concentrations for 24 h. To the culture, 20 μM of the calpain-specific substrate t-BOC-Leu-Met (ThermoFisher) was added and incubated for 30 min at 37° C. with either DMSO or 25 μM of calpain-specific protease inhibitor PD150606 (Sigma). Cells were then analyzed by flow cytometry using 355 nm excitation for substrate (405 nm) and product (430 nm) emission intensity. The ratio of product:substrate was then calculated to reflect calpain activity levels.

### Statistics

For statistical analysis between two groups, the two-tailed unpaired Student's t test was used. When more than two groups were compared, one-way ANOVA was used. Results are expressed as mean ±SEM. The Bonferroni and Dunnett multiple comparison tests were used for post hoc analysis to determine statistical significance between multiple groups. All statistics were calculated using Prism 7 (GraphPad, La Jolla, Calif.) software. Differences among group means were considered significant when the probability value, p, was less than 0.05. Sample size (‘n’) always represents biological replicates. Cochran test was used for exclusion of outliers.

### Results

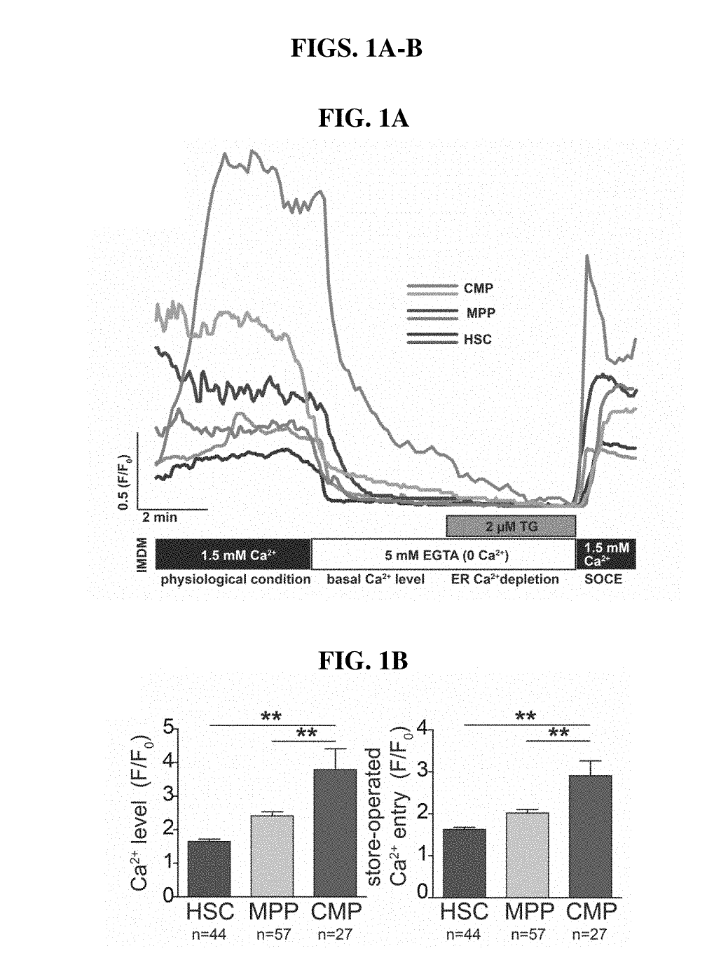

It has previously been shown that the mitochondrial fusion protein, Mfn2, is specifically required for the maintenance of HSCs with extensive lymphoid potential, but not, or less so for the maintenance of myeloid-dominant HSCs⁴. Mfn2 increased buffering of Caᵢ²⁺, (where Caᵢ²⁺ refers to intracellular calcium) an effect mediated through its ER-mitochondria tethering activity,^{5, 6} thereby negatively regulating nuclear translocation and transcriptional activity of Nuclear Factor of Activated T cells (NFAT). Within the HSC compartment, Caᵢ²⁺ was consistently lower in lymphoid-biased CD150^{lo} than in myeloid-biased CD150^{hi} HSCs⁴. During these studies, it was observed using the ratiometric calcium indicator, Indo 1, that, notwithstanding heterogeneity in Caᵢ²⁺ among HSC subpopulations, HSCs overall display exceptionally low Caᵢ²⁺ compared to progenitor populations. To further investigate calcium homeostasis, perfusion Caᵢ²⁺ imaging using FluoForte calcium dye in HSCs, multipotential progenitors (MPPs) and common myeloid progenitors (CMPs) was conducted, where HSCs are defined as Lin⁻Scal⁺Kit⁺CD150⁺CD48⁻Flt3⁻cells, MPPs as Lin⁻Scal⁺Kit⁺CD150⁻CD48+Flt3⁺ and CMPs as lin⁻Scal⁻kit⁺ cells which include common and granulocyte/macrophage progenitors. Compared to CMPs, HSCs, and to lesser extent MPPs had very low Caᵢ²⁺ baseline conditions (FIGS. 1A-1B), confirmed the previous data. Furthermore, store-operated calcium entry (SOCE), the entry of Ca²⁺ from the extracellular space in response to depletion of ER Ca²⁺ stores by thapsigargin,⁷ was higher in MPs than in HSCs and MPPs (FIGS. 1A-1B). HSCs, and to a lesser extent MPPs, therefore have exceptionally low Caᵢ²⁺ and blunted calcium entry.

Standard culture media contain 1.5 to 1.8 mM CaCl₂. As proprietary serum supplements contribute an unknown amount of calcium to the culture media we measured the calcium concentration of commercially available serum free media (SFM) supplements (StemPro34 (Life Technologies) and StemSpan (Stem Cell Technologies)) using the metallochromic indicator Arsenazo III, which absorbs at 650 nm proportional to the amount of Ca²⁺ in solution and is unreactive with Mg²⁺. We found that working preparations of StemPro34 and StemSpan contributed between 0.01-0.05 mM Ca²⁺ to solution, indicating that a minimal concentration of calcium in solution of approximately 0.01-0.05 mM can be achieved without the use of calcium chelators. We therefore cultured purified HSCs in calcium-free DMEM with serum-free supplement, Kit Ligand (KL) and thrombopoietin (TPO), and in the presence of increasing CaCl₂ concentrations up to 2 mM to assess the effect of reduced Caᵢ²⁺ on HSCs in vitro.

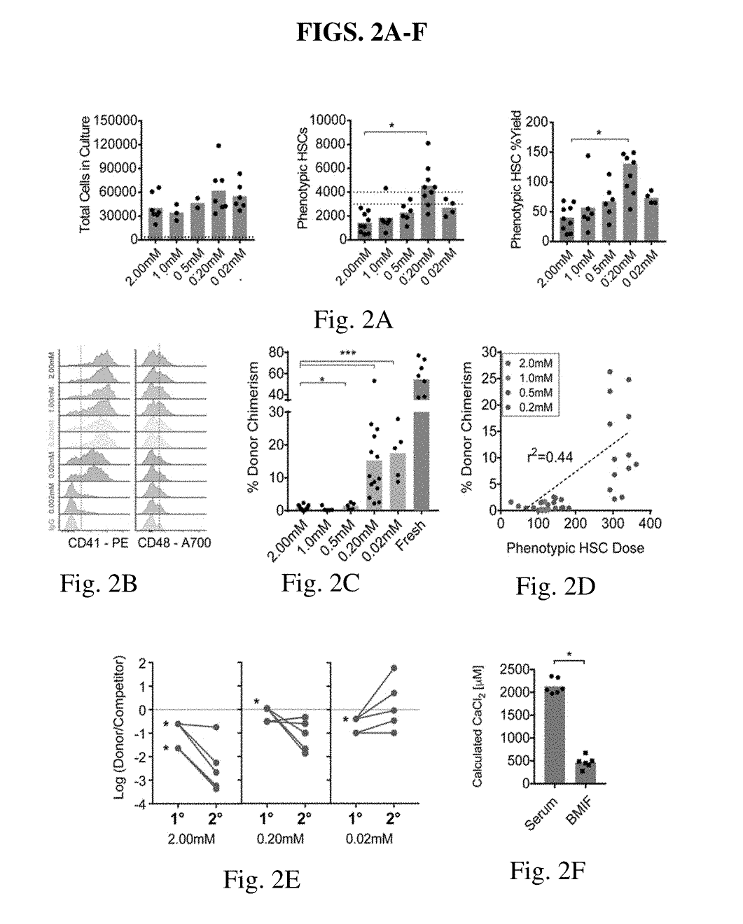

After 2 weeks of culture of purified lin⁻Scal⁺ckit⁺CD150⁺CD48⁻CD41⁻ HSCs total cellular expansion was similar in all conditions. However, fraction and absolute number of HSCs as defined by surface marker phenotype was two- to threefold higher in low than in high calcium media (FIG. 2A). As megakaryocytes are hierarchically and temporally the first lineage generated from HSCs (Adolfsson et al., 2005; Notta et al., 2016; Sawai et al., 2016), we also monitored the appearance of the megakaryocyte marker, CD41. While in the presence of 2.00 mM calcium the majority of the cells in the lin⁻Scal⁺kit⁺ progenitor gate expressed CD41 after two weeks, less than 1% of the cells did so in the presence of 0.02 mM calcium (FIG. 2B). Furthermore, induction of the multipotential progenitor marker (Kiel et al., 2005), CD48, was also attenuated in the presence of low calcium (FIG. 2B). Together, these data indicate that low calcium inhibited differentiation.

To test HSC function, irradiated recipient mice were competitively transplanted with 10% of the cultures, either at initiation or after 2 weeks of culture, and analyzed 15 weeks after transplantation. Donor chimerism from cells cultured in 0.02 mM and 0.2 mM CaCl₂ was ˜10-20-fold higher than that from cells cultured in 2.0 mM CaCl₂, and was approximately one third of that of cells transplanted at initiation of the culture (FIG. 2C). Donor contribution correlated with calculated phenotypic HSC cell dose transplanted (FIG. 2D). Next, we performed secondary transplantations by repopulating lethally irradiated secondary recipients with 10⁶ cells from primary recipients. Because the primary recipients of cells cultured in high calcium showed much lower donor repopulation than those of cells cultured in low calcium, secondary reconstitution was expressed as the log fold change in donor repopulation between secondary and primary recipients to compare the serial transplantation data. Secondary donor repopulation capacity from primary recipients of HSCs from low Caₑ²⁺ cultures was stable or further rose compared to the primary recipients, while that of cells cultured in high calcium declined (FIG. 2E). These findings indicate that low calcium is particularly critical for the functional maintenance of the most primitive HSCs capable of secondary repopulation. We note that when purified CD150^{hi} or CD150^{lo} HSCs were used, only CD150^{hi} cells were maintained, while CD150^{lo} HSC could not be maintained or grown in any condition (not shown), suggesting that the cytokines used are not sufficient to support CD150^{lo} HSCs. While uninformative on the effect of calcium on CD150^{lo}, primarily lymphoid-biased HSCs, these data suggest a profound effect of calcium on the maintenance of CD150^{hi} HSCs.

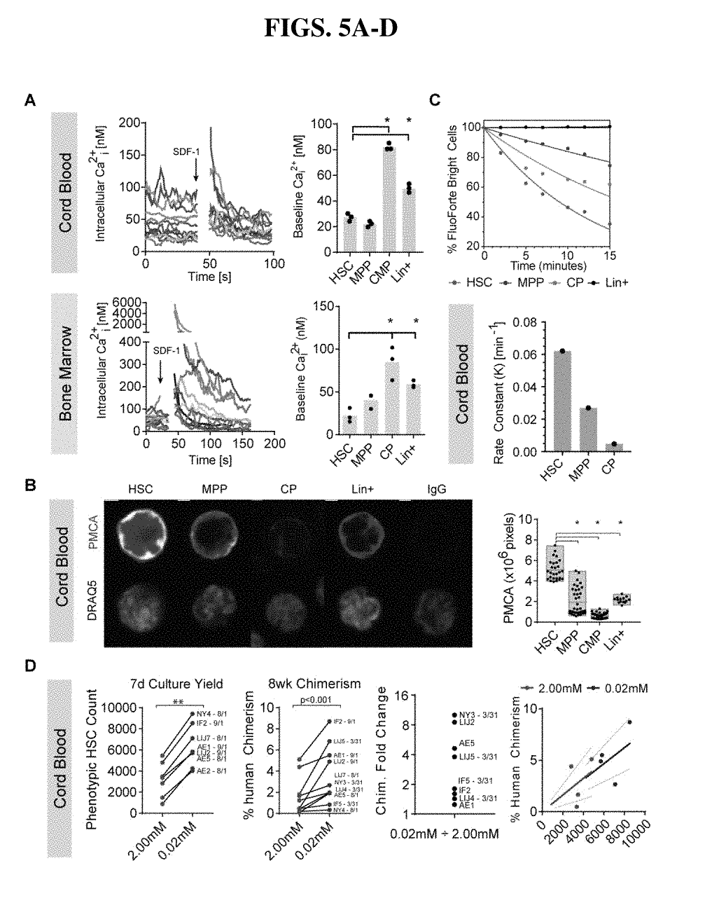

As reduced Caₑ²⁺ enhanced HSC maintenance in vitro we wondered whether the BM would provide a low calcium environment in vivo. It has been reported that around osteoporotic osteoclastic erosion sites in bone, calcium can be as high as 40 mM or 20-fold higher than in serum (Silver et al., 1988), a finding that was used to support the notion that high BM calcium fostered the perinatal transition of hematopoiesis from fetal liver to BM (Adams et al., 2006). In contrast, we found that calcium in the BM interstitial fluid was in fact fourfold lower than in serum (FIG. 2F), a finding possibly explained by the fact that bone is a physiological calcium buffer in steady-state. The low calcium concentration in BM interstitial fluid is consistent with the requirement for a low calcium environment for HSC maintenance in vitro. A similar requirement for low calcium environment were obtained using human cells (FIG. 5D).

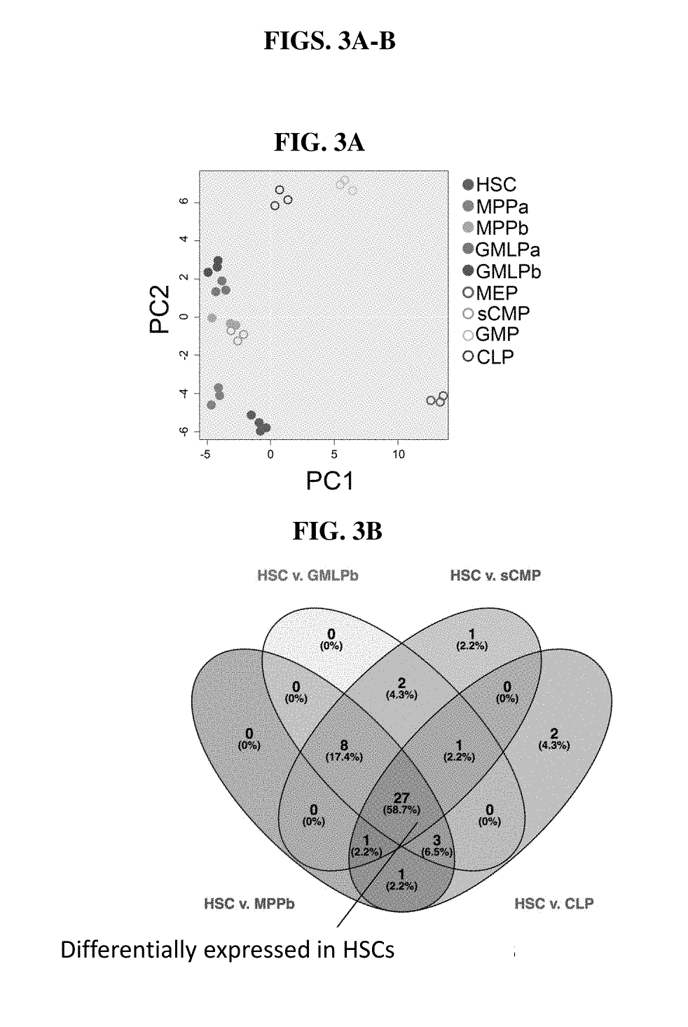

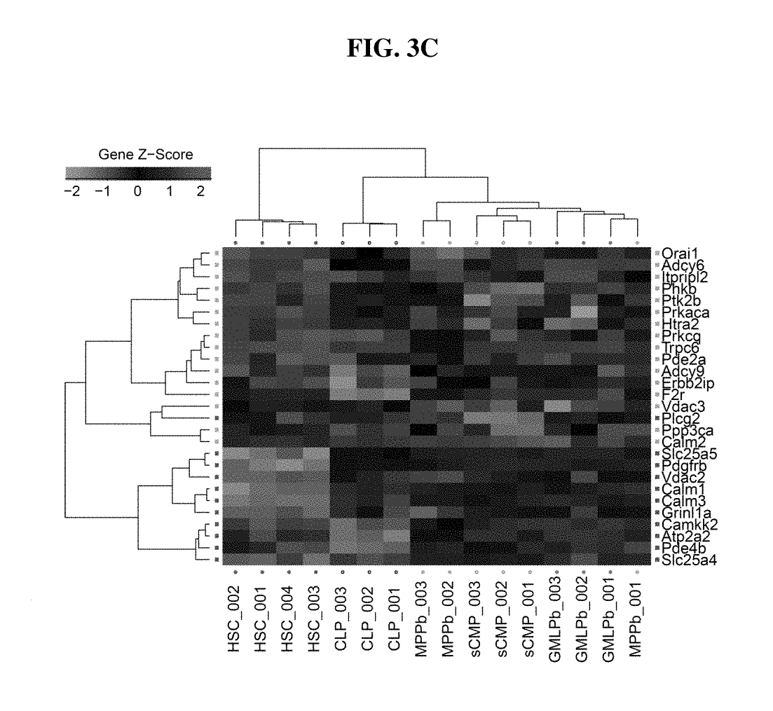

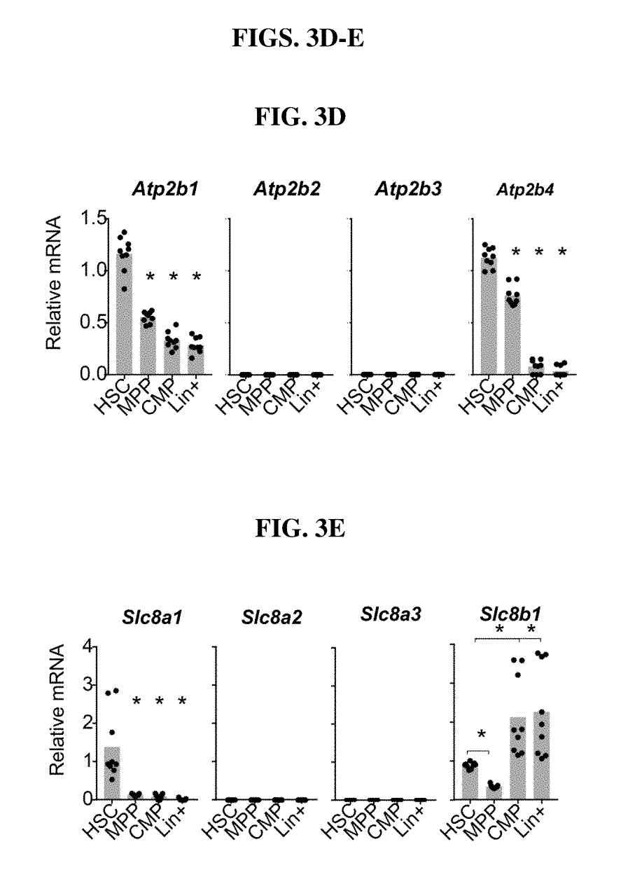

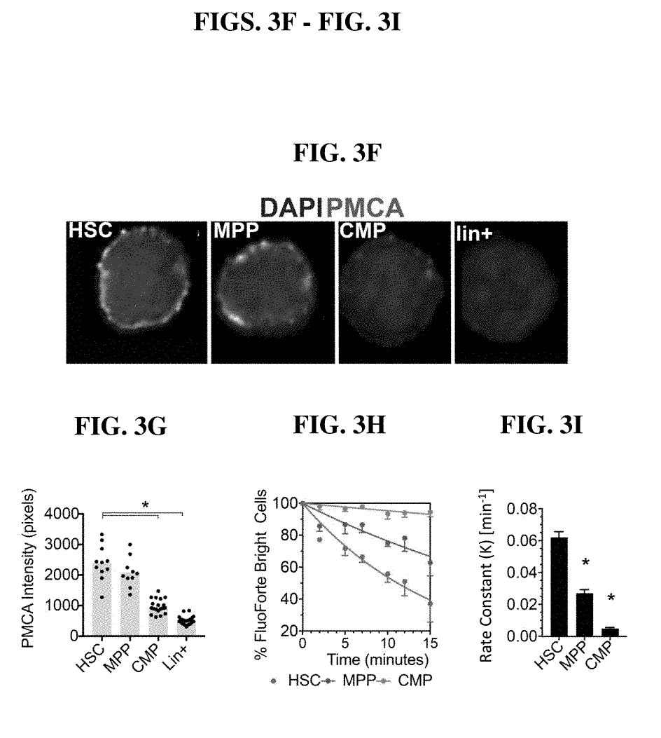

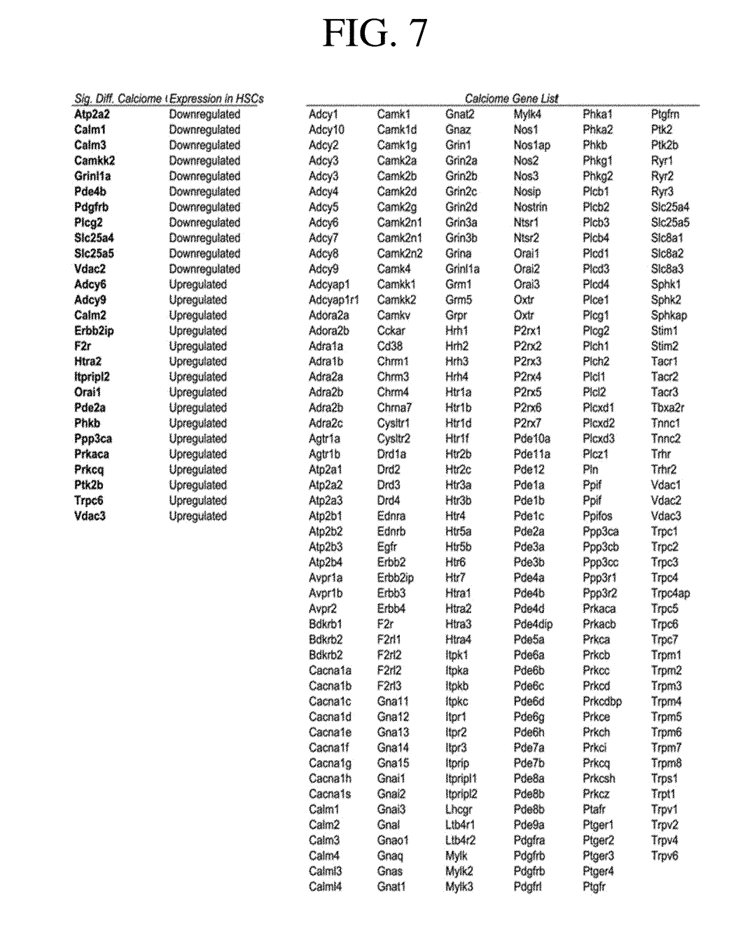

Next, the mechanisms by which HSCs maintain low Caᵢ²⁺ was evaluated. It was previously shown that high Mfn2 expression contributes to low Caᵢ²⁺ in HSCs. However, deletion of Mfn2 only impaired the maintenance of lymphoid-biased HSCs, which appear particularly reliant on low Caᵢ²⁺. To identify further mechanisms underlying low Caᵢ²⁺ in HSCs, calcium in BM interstitial fluid was measured. It has been reported that around osteoporotic osteoclastic erosion sites in bone, calcium can be as high as 40 μM or 20-fold higher than in serum¹¹, a finding that was used to support the notion that HSCs home to BM based on high BM calcium³. It was found however that calcium in the BM interstitial fluid, the small amount of supernatant of spun BM, was in fact fourfold lower than in serum (FIG. 2F), a finding possibly explained by the fact that bone is an organismal calcium buffer. As not only HSCs, but all hematopoietic cells are likely exposed to low BM interstitial fluid calcium, additional mechanism were searched that convey low Caᵢ²⁺ specifically to HSCs. All genes involved in Caᵢ²⁺ regulation and responses were extracted (the ‘calciome’, FIG. 7) according to the GO database from the publicly available GEXC database (gexc.stanford.edu/models/3/genes/). Unsupervised clustering and principal component analysis (FIG. 3A) showed that each progenitor and stem cell subpopulation expresses a distinct calciome signature. Analysis was then focused on those populations in GECX database that are the closest to the populations we considered here (HSCs, MPPs, and GMPb and CMP, which we collectively isolated as MPs) and included common lymphoid progenitors as well. In this analysis, 29 out of 149 ‘calciome’ genes were significantly up or down regulated in HSCs compared to other progenitor populations (FIGS. 3B-3C, FIG. 7). Expression of the mediators of SOCE, the ER-located STIM1, the membrane channel ORAI1,⁷ as well as TRPC6 are highly in expressed HSCs, a finding confirmed by RT-qPCR. This was surprising as SOCE was lower in HSCs and suggested that calcium efflux might be highly active in HSCs. Therefore, expression of PMCA pumps (Atpb1-4), the principal ion channels responsible for extrusion of calcium from non-excitable cells, as well as of Ca²⁺/Na⁺ exchangers (Slc8a1-3, typically expressed in excitable cells) and the mitochondrial Ca²⁺ /Na+exchanger, Slcb8b1 were measured. Although not significantly differentially expressed among progenitor populations by microarray in the GEXC database, two (Atp2b1 and Atp2b4) of the four genes encoding PMCA pumps (FIG. 3D) and one Ca²⁺/Na⁺ exchanger (S1c8a1) (FIG. 3E) were selectively expressed in HSCs. Immunofluorescence using a pan-PMCA antibody confirmed increased expression in HSCs, with a progressive decrease in progenitors and lineage positive cells (FIGS. 3F-3G). Furthermore, functional flow cytometry calcium efflux assay showed that, consistent with the mRNA and protein expression data for PMCA pumps, calcium efflux was highest in HSCs, followed by MPPs and CMPs (FIGS. 3H-3I). Multiple mechanisms, including low BM interstitial fluid calcium, high efflux mediated several pumps as well as mitochondrial hyperfusion therefore contribute to low Caᵢ²⁺ in HSCs.

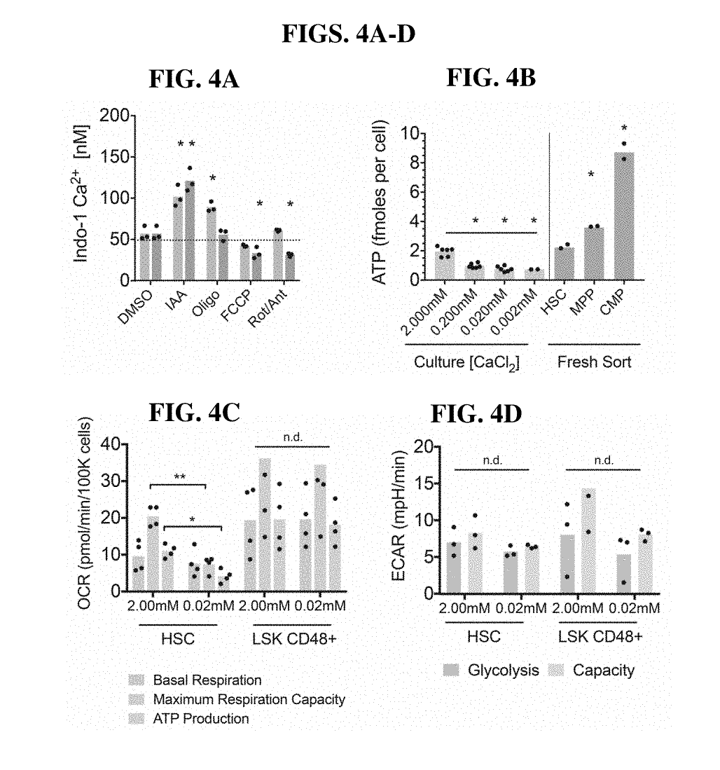

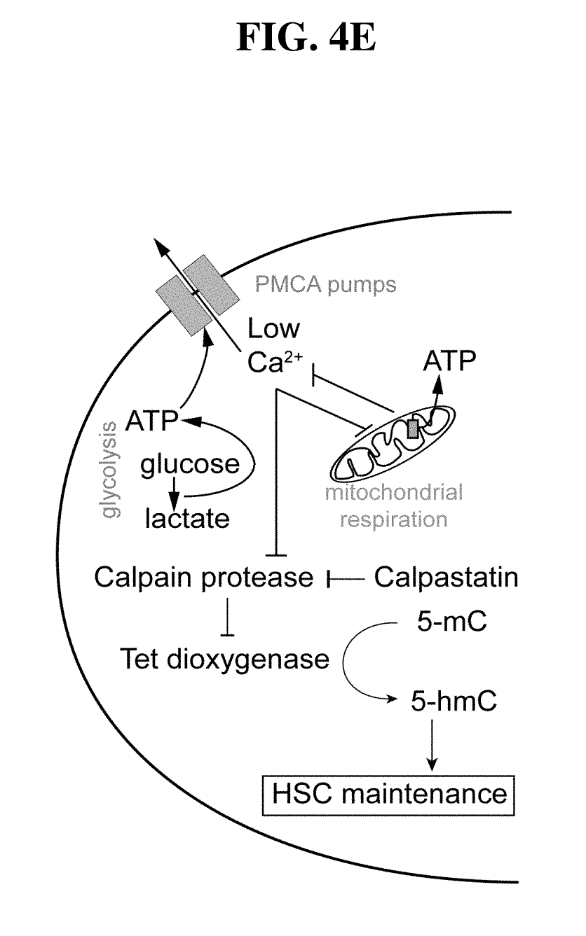

In addition to low Caᵢ²⁺, glycolytic ATP production is a well-established feature of HSCs and critical to HSC maintenance, while increased mitochondrial respiration is required for differentiation. Therefore, it was examined whether HSC metabolism and Caᵢ²⁺ interact. Interestingly in this context, PMCA pumps predominantly use glycolysis as a source of ATP in several cell types¹²⁻¹⁶, including red blood cells where components of the glycolytic pathway are physically associated with the cell membrane, while ATP pools are present close to cation channels¹⁷⁻¹⁹. Purified HSCs were incubated with an inhibitor of glycolysis (iodoacetic acid) and several inhibitors of mitochondrial respiration (the ATP synthase inhibitor oligomycin, the uncoupler FCCP, and the complex I inhibitors rotenone/antimycin). Inhibition of glycolysis, but not inhibition of mitochondrial respiration increased Caᵢ²⁺ (FIG. 4A). In fact, at higher doses of the mitochondrial inhibitors, FCCP and oligomycin/antimycin, a further reduction in Caᵢ²⁺ was observed (FIG. 4A). As calcium positively regulates the activity of at least three enzymes of the Krebs cycle, we next assessed the effect of low calcium on HSC metabolism. Incubation of enriched HSCs in low Caₑ²⁺ decreased mitochondrial calcium concentration as measured by mitochondrial calcium probe, Rhod2, and decreased intracellular ATP levels (FIG. 4B) suggesting repression of respiration. Seahorse extracellular flux analysis confirmed HSCs in low Caₑ²⁺ showed reduced maximum mitochondrial respiratory capacity (FIG. 4C) and mitochondrial ATP production, but did not affect glycolysis (FIG. 4D). In contrast, in MPPs low Caₑ²⁺ did not significantly affect mitochondrial function. Taken together, these findings indicate that glycolysis in HSC fuels calcium efflux from HSCs, thus lowering Caᵢ² and reducing mitochondrial respiration, which in turn further promotes low Caᵢ²⁺ specifically in HSCs.

It has been shown here that HSCs are endowed, through multiple mechanisms, with exceptionally low Caᵢ²⁺ and culture in low Caₑ²⁺ strongly enhances their maintenance in vitro. Glycolysis is required to maintain low HSC Caᵢ²⁺ and is critical to HSC function. On the other hand, mitochondrial respiration in HSCs regulates and is regulated by Caᵢ²⁺ and is required for differentiation. It this therefore possible that HSC metabolism is not, as is widely believed, governed by presumed BM hypoxia, but aimed at maintaining low Caᵢ²⁺. Importantly, this work provides a physiological approach for the development of strategies to maintain HSCs in vitro.

Human HSCs display low Caᵢ²⁺, and culture in low calcium enhances their maintenance in vitro.

Next, the mouse data in human cord blood stem cells was verified. Similar to the mouse, Caᵢ²⁺ as measured by flow cytometry using the radiometric dye, Indol, was the lowest in HSCs, and increased with differentiation (FIG. 5A). Furthermore, both activity and expression of PMCA efflux pumps was the highest in HSC, once again paralleling the data in the mouse (FIGS. 5B-5C). Culture of human HSCs (lin−CD34+CD38−CD90+) in low calcium resulted in better maintenance of HSCs as defined by phenotype (FIG. 5D), and better repopulation ability in immunodeficient NSG mice after 4 weeks (FIG. 5D). Though longer follow-up of the mice and further optimization of low calcium culture conditions is in order, these data indicate the similar to the mouse, low calcium is required for optimal maintenance of HSCs in vitro.

Finally, whether human HSCs are also endowed with low Caᵢ²⁺ and whether low calcium culture conditions would also support improved maintenance of human HSCs in vitro were examined. Similar to the mouse, Caᵢ²⁺ was lower (FIG. 5A), PMCA expression (FIG. 5B) and PMCA pump efflux activity (FIG. 5C) were higher in HSCs than in progenitor and lint cells. After culture for 7 days in low calcium the number of phenotypically defined HSCs was significantly higher than after culture in high calcium (FIG. 5D). Furthermore, 9 weeks after transplantation into sublethally irradiated, immunodeficient NSG mice, repopulation with human cells was 2 to tenfold higher in recipients of HSC cultured in low compared to high calcium (FIG. 5D). Similar to mouse HSCs, therefore, human HSCs have lower Caᵢ²⁺ than progenitors, and a reduced calcium environment promotes their maintenance in culture.

Calpain inhibition is involved in the effect of low Caₑ²⁺

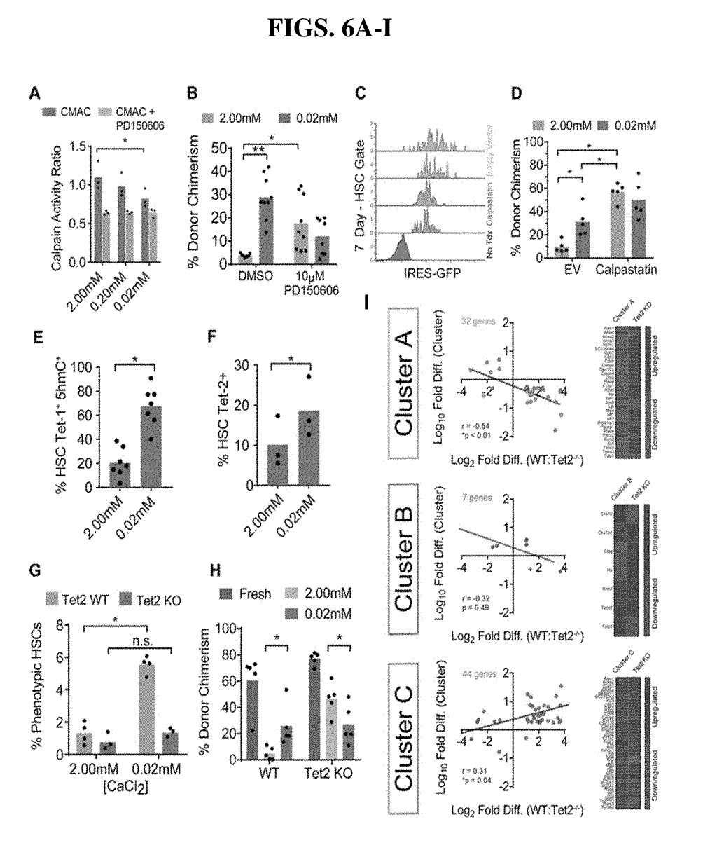

Next the downstream mechanisms underlying the effect of low calcium on HSC maintenance was examined. Calpains are a family of 14 widely expressed proteolytic enzymes (Storr et al., 2011), of which according to the GEXC database (https://gexc.riken.jp/) at least three (Capn2, 5 and 7) and a small regulatory subunit required for the activity of some calpains (Capns1) are expressed in HSCs. Calpain activity was detected in purified HSCs by a flow cytometric assay (Niapour and Berger, 2007), and was dose-dependently reduced after culture in low calcium media (FIG. 6A). To evaluate whether calpain inhibition is involved in the effect of low calcium conditions on HSC maintenance, we cultured HSC in high (2.0 mM) and low (0.02 mM) calcium in the presence of the small molecule calpain inhibitor, PD150606 (Wang et al., 1996), which was replenished every other day. After 2 weeks of culture, we transplanted 10% of the culture in competitive repopulations assays. These experiments showed that addition of PD150606 to high calcium cultures increased competitive repopulation capacity, while no effect was noted in low calcium cultures (FIG. 6B). These findings suggest that inhibited calpain activity is involved in the effect of low calcium on the functional maintenance of HSCs. To further substantiate this notion and since small molecules may have off-target or toxic effects, we lentivirally overexpressed the endogenous calpain inhibitor, calpastatin (Cast) (Storr et al., 2011) during in vitro culture (FIG. 6C). Subsequent competitive transplantation revealed strongly enhanced HSC maintenance in high calcium cultures to a level that was even higher than that of low calcium cultures of empty vector-transduced cells (FIG. 6D).

HSCs cultured for 2 weeks in low calcium showed by flow cytometry significantly higher global 5 hmC content and TET1 (FIG. 6E) and TET2 (FIG. 6F) expression than HSCs cultured in high calcium, indicating stabilization of all expressed TET isoforms in low calcium conditions.

As TET2, but not TET1, is required for normal HSC function, the effect of calcium on the maintenance of Tet2−/− HSCs was examined. In contrast to its effect on the maintenance of wt HSCs, calcium did not affect the maintenance of phenotypically defined Tet2−/− HSCs (FIG. 6G) such that even in low calcium cultures, a similar number of phenotypically defined Tet2−/− HSCs were present as in high calcium for both wt and Tet2−/− HSCs. Next, 5% of the cells were used in competitive transplantation assays. In these experiments, low calcium culture media did not increase subsequent competitive repopulation capacity of Tet2−/− cells compared to high calcium culture media, in sharp contrast to the effect of calcium on the maintenance of wt HSCs. In fact, low calcium had a marginally suppressive effect on HSC maintenance compared to high calcium (FIG. 6H). Repopulating Tet2−/− HSCs are therefore impervious to the enhancing effect of low calcium on their maintenance.

To further strengthen this point, whether a TET2 gene signature was differentially expressed in high and low calcium cultures was analyzed. The scRNA-seq gene expression clusters that were differentially expressed in high and low calcium cultures (FIG. 2) were cross-referenced with publicly available data generated from purified wt and Tet2−/− HSCs, which identified 889 differentially expressed genes (Zhang et al., 2016). We found that ˜15% of the differentially expressed genes within Cluster A and C were present in the Tet2 gene signature, which is significantly more than expected. For the overlapping genes, correlation analysis of fold difference in the expression between wt and Tet2−/− HSCs with differential expression among clusters revealed that cluster C genes positively associated with the Tet2 gene expression signature (r=0.31, p=0.04) while Cluster A genes showed a negative correlation (r=−0.54, p<0.01) (FIG. 6I). As cluster C was enriched in low calcium cultures, these data indicate that low calcium culture conditions maintain a transcriptional profile that is reciprocally altered in the absence of TET2 expression, and therefore lend further support to the notion that TET2 plays a role in the effect of low calcium on HSC maintenance.

These data indicate that calpain inhibition is at least one of the downstream mechanisms underlying the enhanced HSC maintenance induced by a low calcium environment. Additionally, these data illustrate that anything that inhibits calpains in fact maintains HSCs. Included among calpain inhibitors that are expected to maintain or enhance HSC maintenance in low calcium environments, are genetic methods for overexpression of calpastatin (Cast) and small molecule inhibitors of calpains, and genetic modifications that reduce calpain levels.

These data demonstrate that low Caᵢ²⁺ is critical for HSC maintenance and that the specific metabolic wiring of HSCs, high glycolysis and low mitochondrial respiration, is required to achieve low Caᵢ²⁺, and is maintained by low Caᵢ²⁺. This work suggests that glycolysis and impaired respiration in HSCs may not be driven by presumed bone marrow hypoxia, but are hard-wired in order to maintain low Caᵢ²⁺, while low Caᵢ²⁺ in turn maintains the metabolic state of HSC through a positive feedback.

As will be apparent to one of ordinary skill in the art from a reading of this disclosure, further embodiments of the present invention can be presented in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. Such equivalents are considered to be within the scope of this invention. Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention. The scope of the invention is as set forth in the appended claims and equivalents thereof, rather than being limited to the examples contained in the foregoing description.

### REFERENCES

1. Orkin, S. H. & Zon, L. I. Hematopoiesis: an evolving paradigm for stem cell biology. Cell 132, 631-644 (2008).
2. Rossi, L. et al. Less is more: unveiling the functional core of hematopoietic stem cells through knockout mice. Cell Stem Cell 11, 302-317 (2012).
3. Kondo, M. et al. Biology of hematopoietic stem cells and progenitors: implications for clinical application. Annual review of immunology 21, 759-806 (2003).
4. Himburg, H. A. et al. Pleiotrophin regulates the expansion and regeneration of hematopoietic stem cells. Nat Med 16, 475-482 (2010).
5. Zhang, C. C. et al. Angiopoietin-like proteins stimulate ex vivo expansion of hematopoietic stem cells. Nat Med 12, 240-245 (2006).
6. Zhang, C. C., Kaba, M., lizuka, S., Huynh, H. & Lodish, H. F. Angiopoietin-like 5 and IGFBP2 stimulate ex vivo expansion of human cord blood hematopoietic stem cells as assayed by NOD/SCID transplantation. Blood 111, 3415-3423 (2008).
7. Drake, A. C. et al. Human CD34+CD133+ hematopoietic stem cells cultured with growth factors including Angptl5 efficiently engraft adult NOD-SCID Il2rgamma−/− (NSG) mice. PLoS One 6, e18382 (2011).
8. Hoggatt, J., Singh, P., Sampath, J. & Pelus, L. M. Prostaglandin E2 enhances hematopoietic stem cell homing, survival, and proliferation. Blood 113, 5444-5455 (2009).
9. North, T. E. et al. Prostaglandin E2 regulates vertebrate haematopoietic stem cell homeostasis. Nature 447, 1007-1011 (2007).
10. Goessling, W. et al. Prostaglandin E2 enhances human cord blood stem cell xenotransplants and shows long-term safety in preclinical nonhuman primate transplant models. Cell Stem Cell 8, 445-458 (2011).
11. Goessling, W. et al. Genetic interaction of PGE2 and Wnt signaling regulates developmental specification of stem cells and regeneration. Cell 136, 1136-1147 (2009).
12. Hagedorn, E. J., Durand, E. M., Fast, E. M. & Zon, L. I. Getting more for your marrow: boosting hematopoietic stem cell numbers with PGE2. Experimental cell research 329, 220-226 (2014).
13. Zhang, Y. & Gao, Y. Novel chemical attempts at ex vivo hematopoietic stem cell expansion. International journal of hematology 103, 519-529 (2016).
14. Boitano, A. E. et al. Aryl hydrocarbon receptor antagonists promote the expansion of human hematopoietic stem cells. Science 329, 1345-1348 (2010).
15. Fares, I. et al. Cord blood expansion. Pyrimidoindole derivatives are agonists of human hematopoietic stem cell self-renewal. Science 345, 1509-1512 (2014).
16. Nishino, T. et al. Ex vivo expansion of human hematopoietic stem cells by a small-molecule agonist of c-MPL. Experimental hematology 37, 1364-1377 e1364 (2009).
17. Huang, J., Nguyen-McCarty, M., Hexner, E. O., Danet-Desnoyers, G. & Klein, P. S. Maintenance of hematopoietic stem cells through regulation of Wnt and mTOR pathways. Nat Med 18, 1778-1785 (2012).
18. Nishino, T. et al. Ex vivo expansion of human hematopoietic stem cells by garcinol, a potent inhibitor of histone acetyltransferase. PLoS One 6, e24298 (2011).
19. de Lima, M. et al. Transplantation of ex vivo expanded cord blood cells using the copper chelator tetraethylenepentamine: a phase I/II clinical trial. Bone marrow transplantation 41, 771-778 (2008).
20. Wagner, J. E., Jr. et al. Phase I/II Trial of StemRegenin-1 Expanded Umbilical Cord Blood Hematopoietic Stem Cells Supports Testing as a Stand-Alone Graft. Cell Stem Cell 18, 144-155 (2016).
21. Doulatov, S., Notta, F., Laurenti, E. & Dick, J. E. Hematopoiesis: a human perspective. Cell Stem Cell 10, 120-136 (2012).
22. Rentas, S. et al. Musashi-2 attenuates AHR signalling to expand human haematopoietic stem cells. Nature 532, 508-511 (2016).
23. Bernitz, J. M., Kim, H. S., MacArthur, B., Sieburg, H. & Moore, K. Hematopoietic Stem Cells Count and Remember Self-Renewal Divisions. Cell 167, 1296-1309 e1210 (2016).
24. Wang et al (1996) An alpha-mercaptoacrylic acid derivative is a selective nonpeptide cell-permeable calpain inhibitor and is neuroprotective. Proc. Natl. Acad. Sci. U.S.A. 93: 6687.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GenelD entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GenelD entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

What is claimed is:

1. Method of maintaining hematopoietic stem cells (HSCs) in a multipotent state, the method comprising:
obtaining a population of HSCs; and
culturing the population in a low calcium medium under conditions, in vitro, to maintain multipotency of the cultured population for at least 7 days, wherein the low calcium medium comprises a calcium concentration of below 1.5 mM.

2. The method of claim 1, wherein the cultured population retains a capacity for self-renewal and multilineage differentiation.

3. The method of claim 1, wherein the calcium concentration is between about 0.01 mM and 1.5 mM.

4. The method of claim 1, wherein the calcium concentration is between about 0.002 mM and 0.01 mM.

5. The method of claim 1, wherein the cultured population exhibits Lin−Scal+Kit+CD150+ CD48−CD41−Flt3−CD34− as a surface marker profile.

6. The method of claim 1, wherein culturing comprises culturing the population in a low calcium medium under conditions to maintain multipotency of the cultured population for at least 2 weeks.

7. The method of claim 1, wherein the low calcium medium lacks alanine, asparagine, glutamic acid or aspartic acid, or a combination thereof.

8. The method of claim 7, wherein the low calcium medium lacks alanine, asparagine, glutamic acid and aspartic acid.

9. A culture medium for maintaining HSCs comprising:
one or more inorganic salts;
one or more amino acids;
one or more vitamins;
one or more saccharides;
optionally one or more trace elements, or iron selenite, insulin, transferrin, lipids and combinations thereof,
optionally one or more calpain inhibitors, and
calcium between 0.002 mM and 1.2 mM concentration.

10. The culture medium of claim 9, wherein the one or more inorganic salts comprise ferric nitrate, magnesium sulfate, potassium chloride, sodium chloride or sodium phosphate monobasic, or a combination thereof.

11. The culture medium of claim 9, wherein the one or more amino acids comprise 1-arginine, 1-cystine, 1-glutamine, glycine, 1-histidine, 1-isoleucine, 1-lysine, 1-methionine, 1-phenylalanine, 1-serine, 1-threonine, 1-tryptophan, 1-tyrosine or 1-valine or a combination thereof.

12. The culture medium of claim 9, wherein the one or more vitamins comprises choline chloride, folic acid, myo-inositol, niacinamide, d-pantothenic acid, pyridoxal, riboflavin or thiamine, or a combination thereof.

13. The culture medium of claim 9, wherein the one or more saccharides is d-glucose.

14. The culture medium of claim 9, further comprising pyruvic acid.

15. The method of claim 1 wherein culturing occurs at least 14 days.

16. A kit comprising the culture medium of claim 9.

17. The kit of claim 16, further comprising one or more additional components selected from the group consisting of culture media, buffers, growth factors, and optionally cell lines.

18. The method of claim 1, further comprising culturing the cells under conditions which inhibit calpain.

19. The method of claim 18, wherein inhibiting calpain comprises including an inhibitor of calpain.

20. The method of claim 19, wherein the inhibitor of calpain comprises PD150606.

21. The method of claim 18, wherein the cells have been genetically modified to produce a reduced level of calpain.

22. The culture medium of claim 9, wherein the inhibitor of calpain comprises PD150606.

23. The culture medium of claim 9, wherein the inhibition of calpain comprises genetically modifying cells to produce a reduced level of calpain.
